SEQUENCE LISTING (5 sequences)

SEQ ID NO: 1 - protein, 1936 aa, Bacillus circulans
MSSRLKRKCSMLLTFTMIFQLLGLFLFKGEIVSASIRQDPTTGNYYKNVPLVGADFDDASNSNIVAKGTWDDNTAPLNTFFVDKGTATDGGFTTARITTVTDQVYEGGSLQFGDGSTYPINLNYKVDGLEVGATYRLSAYMKLFPGYPAKGGQFGVKNHDTANYTTGGETKSVNFSTVTADWKEYSVTFTPTYPHAKIFFWGSNNLPKVLVDKLRLEKVLEHPGPAAPAVTADDVNNIVVGIDETMEYNINGAGWVAYKEYAKPDLKGDLIVQIRVKETLNTLAGEVTTLTFTAQNDPAPGQPEQLLLKDGDFEAGAASVTTDTNVQNQFFSKNNQYEIVTGDTASGQYALKLRSPETIGYHKTDLKPSTKYQISFMAKVGSASQKLSFRISGYKNDNPYDLDNVMNYIEHTQMKNTGWSRFYYDLETGPSATSAFIDFSTAAGSTAWIDDVKLVEQGPADPPVTEPTLSRGSRLFLEKGLQIQSWVPTDVAYATRKWMKPPTAEEIVDLGLTTVQYNDAPNYSKTLHEEYKKLQQTNPSLPDLKWGVAFGPNANHLSSSYFDSETIAKHDPNKTGAPTEEQKARGFLTPDQLANVQNLNNIGFGDEEDYSDTLTQTLKEWFEVSKKHYPNVLVHHNEVGNTPPPTMSLISTFNENMLRKYMRTAKPDFITYDMYYFRENRQSSEVGGTVIPFYDDLNRYRKVASEGYDGSGLSPIPFGTYLQGWRTGPGAATYEKRGDGWYEITESQAYLSAFANWTFGAKWLSMFRWIEDTPGYLFSDYRPDEDGNWPKYHIYGQYKEMFRQSKNLGEHLIRINNKDVVIVPGQHMKDGQITKNNRPKDNPEWTKSGDRAFIDSLEISNLGKTNHSLKGDVFIGYFDPLPGIDTTQFFTSTAPKYFMLLNGLTSGQGLPAEEQTGSSYETRQEIKVTFDLSGGQARADQLRKVSRLTGELVAAPLKDLGNGKYEMTVVLGGGMADLYFWELGSLNTGNSKPVVADTPHDVRLTGDPKYAKNREIRDLTGKTVTVGWIKDTYSPVPQPLIHYNFSFTKDQNGKLQPMKNPDILSYFTRYYENTLWNKRVERIQKESNVKLEFVADIAWTKQELMDNIRKVKEGQTVDGMPDILIVPDEWTWSGLIQNEMILPASSFSEFDFTERKWNKSYKAMTTWKDQIYGMYAGPTMNSTGLFVNKALQASIGVTDDLMALQQNNAWDWNKLREVASAFQASANREGKYLLAGTDELFKQMVYANGAARGSVGGAMNQEFDLTSSSFREAAELYSELHAAGLIAAKPEGATDDWYVEQFSKNNILFLALPYQQTVDKLKFSYTNQDAVIEMKEGSFLGQPALIPTIVDAYETAYPDGIYKMAQGDWVFLMFPKGPSATGYAAMIDNPAYPVLLSSSANPADAAYVWNILSHEFEGVAYDRFLKLYLNQREVDKTTLKRIGLKEGVWDSYSGTGAWEQVIKPGVLPMLQAGVIDEAKLAELSVEAASYVTNNMTKPAQPGEEPGEEPGEQPGEQPGEQPGEQPGEQPGEQPGEQPGEQPGEQPGAGNGSENQGGNEDQGGNGSQGGNGPKPEKIIVKPGELIAVEGKVTIVVPAGATEIVLPPQAAELPQQHKVELKTDRVTLEVPSGLLKKLASRIADKDVSISLKAAPLTAAQAKDAISKNKSVSPSAITLAGGVYDFKLSAAGANGSYAELSEFDQPITISLKIESGVNPEQVGIYYISGNGKLDYIGGEYRDGELAAEVTHFSQYAVLKVVKVFDDVPAGHWAEGVISKLTSRLMVDGTSETTFEPERVVTRAEFTALLARALKLTAGGTPTFADVKAGDWYADAVTAAVEAGIAEGKSAGQFEPQARITREEMVVMTMRAYNKAKDKGPSTGVEASFTDENQISAWAVEQVKAAAALQLIQGRAQGKFEPQGTATRAEAVQVIFNMLLK

SEQ ID NO: 2 - protein, 1468 aa, Artificial Sequence
  Description of Artificial Sequence Synthetic polypeptide
SIRQDPTTGNYYKNVPLVGADFDDASNSNIVAKGTWDDNTAPLNTFFVDKGTATDGGFTTARITTVTDQVYEGGSLQFGDGSTYPINLNYKVDGLEVGATYRLSAYMKLFPGYPAKGGQFGVKNHDTANYTTGGETKSVNFSTVTADWKEYSVTFTPTYPHAKIFFWGSNNLPKVLVDKLRLEKVLEHPGPAAPAVTADDVNNIVVGIDETMEYNINGAGWVAYKEYAKPDLKGDLIVQIRVKETLNTLAGEVTTLTFTAQNDPAPGQPEQLLLKDGDFEAGAASVTTDTNVQNQFFSKNNQYEIVTGDTASGQYALKLRSPETIGYHKTDLKPSTKYQISFMAKVGSASQKLSFRISGYKNDNPYDLDNVMNYIEHTQMKNTGWSRFYYDLETGPSATSAFIDFSTAAGSTAWIDDVKLVEQGPADPPVTEPTLSRGSRLFLEKGLQIQSWVPTDVAYATRKWMKPPTAEEIVDLGLTTVQYNDAPNYSKTLHEEYKKLQQTNPSLPDLKWGVAFGPNANHLSSSYFDSETIAKHDPNKTGAPTEEQKARGFLTPDQLANVQNLNNIGFGDEEDYSDTLTQTLKEWFEVSKKHYPNVLVHHNEVGNTPPPTMSLISTFNENMLRKYMRTAKPDFITYDMYYFRENRQSSEVGGTVIPFYDDLNRYRKVASEGYDGSGLSPIPFGTYLQGWRTGPGAATYEKRGDGWYEITESQAYLSAFANWTFGAKWLSMFRWIEDTPGYLFSDYRPDEDGNWPKYHIYGQYKEMFRQSKNLGEHLIRINNKDVVIVPGQHMKDGQITKNNRPKDNPEWTKSGDRAFIDSLEISNLGKTNHSLKGDVFIGYFDPLPGIDTTQFFTSTAPKYFMLLNGLTSGQGLPAEEQTGSSYETRQEIKVTFDLSGGQARADQLRKVSRLTGELVAAPLKDLGNGKYEMTVVLGGGMADLYFWELGSLNTGNSKPVVADTPHDVRLTGDPKYAKNREIRDLTGKTVTVGWIKDTYSPVPQPLIHYNFSFTKDQNGKLQPMKNPDILSYFTRYYENTLWNKRVERIQKESNVKLEFVADIAWTKQELMDNIRKVKEGQTVDGMPDILIVPDEWTWSGLIQNEMILPASSFSEFDFTERKWNKSYKAMTTWKDQIYGMYAGPTMNSTGLFVNKALQASIGVTDDLMALQQNNAWDWNKLREVASAFQASANREGKYLLAGTDELFKQMVYANGAARGSVGGAMNQEFDLTSSSFREAAELYSELHAAGLIAAKPEGATDDWYVEQFSKNNILFLALPYQQTVDKLKFSYTNQDAVIEMKEGSFLGQPALIPTIVDAYETAYPDGIYKMAQGDWVFLMFPKGPSATGYAAMIDNPAYPVLLSSSANPADAAYVWNILSHEFEGVAYDRFLKLYLNQREVDKTTLKRIGLKEGVWDSYSGTGAWEQVIKPGVLPMLQAGVIDEAKLAELSVEAASYVTNNMTKPAQPG

SEQ ID NO: 3 - protein, 1356 aa, Artificial Sequence
  Description of Artificial Sequence Synthetic polypeptide
SIRQDPTTGNYYKNVPLVGADFDDASNSNIVAKGTWDDNTAPLNTFYPHAKIFFWGSNNLPKVLVDKLRLEKVLEHPGPAAPAVTADDVNNIVVGIDETMEYNINGAGWVAYKEYAKPDLKGDLIVQIRVKETLNTLAGEVTTLTFTAQNDPAPGQPEQLLLKDGDFEAGAASVTTDTNVQNQFFSKNNQYEIVTGDTASGQYALKLRSPETIGYHKTDLKPSTKYQISFMAKVGSASQKLSFRISGYKNDNPYDLDNVMNYIEHTQMKNTGWSRFYYDLETGPSATSAFIDFSTAAGSTAWIDDVKLVEQGPADPPVTEPTLSRGSRLFLEKGLQIQSWVPTDVAYATRKWMKPPTAEEIVDLGLTTVQYNDAPNYSKTLHEEYKKLQQTNPSLPDLKWGVAFGPNANHLSSSYFDSETIAKHDPNKTGAPTEEQKARGFLTPDQLANVQNLNNIGFGDEEDYSDTLTQTLKEWFEVSKKHYPNVLVHHNEVGNTPPPTMSLISTFNENMLRKYMRTAKPDFITYDMYYFRENRQSSEVGGTVIPFYDDLNRYRKVASEGYDGSGLSPIPFGTYLQGWRTGPGAATYEKRGDGWYEITESQAYLSAFANWTFGAKWLSMFRWIEDTPGYLFSDYRPDEDGNWPKYHIYGQYKEMFRQSKNLGEHLIRINNKDVVIVPGQHMKDGQITKNNRPKDNPEWTKSGDRAFIDSLEISNLGKTNHSLKGDVFIGYFDPLPGIDTTQFFTSTAPKYFMLLNGLTSGQGLPAEEQTGSSYETRQEIKVTFDLSGGQARADQLRKVSRLTGELVAAPLKDLGNGKYEMTVVLGGGMADLYFWELGSLNTGNSKPVVADTPHDVRLTGDPKYAKNREIRDLTGKTVTVGWIKDTYSPVPQPLIHYNFSFTKDQNGKLQPMKNPDILSYFTRYYENTLWNKRVERIQKESNVKLEFVADIAWTKQELMDNIRKVKEGQTVDGMPDILIVPDEWTWSGLIQNEMILPASSFSEFDFTERKWNKSYKAMTTWKDQIYGMYAGPTMNSTGLFVNKALQASIGVTDDLMALQQNNAWDWNKLREVASAFQASANREGKYLLAGTDELFKQMVYANGAARGSVGGAMNQEFDLTSSSFREAAELYSELHAAGLIAAKPEGATDDWYVEQFSKNNILFLALPYQQTVDKLKFSYTNQDAVIEMKEGSFLGQPALIPTIVDAYETAYPDGIYKMAQGDWVFLMFPKGPSATGYAAMIDNPAYPVLLSSSANPADAAYVWNILSHEFEGVAYDRFLKLYLNQREVDKTTLKRIGLKEGVWDSYSGTGAWEQVIKPGVLPMLQAGVIDEAKLAELSVEAASYVTNNMTKPAQPG

SEQ ID NO: 4 - protein, 1400 aa, Artificial Sequence
  Description of Artificial Sequence Synthetic polypeptide
SIRQDPTTGNYYKNVPLVGADFDDASNSNIVAKGTWDDNTAPLNTFFVDKGTATDGGFTTARITTVTDQVYEGGSLQFGDGSTYPINLNYKVDGLEVGATYRLSAYMKLFPGYPAKGGQFGVKNHDTANYTTGGETKSVNFSTVTADWKEYSVTFTPTYPHAKIFFWGSNNLPKVLVDKLRLEKVLEHPGPAQNDPAPGQPEQLLLKDGDFEAGAASVTTDTNVQNQFFSKNNQYEIVTGDTASGQYALKLRSPETIGYHKTDLKPSTKYQISFMAKVGSASQKLSFRISGYKNDNPYDLDNVMNYIEHTQMKNTGWSRFYYDLETGPSATSAFIDFSTAAGSTAWIDDVKLVEQGPADPPVTEPTLSRGSRLFLEKGLQIQSWVPTDVAYATRKWMKPPTAEEIVDLGLTTVQYNDAPNYSKTLHEEYKKLQQTNPSLPDLKWGVAFGPNANHLSSSYFDSETIAKHDPNKTGAPTEEQKARGFLTPDQLANVQNLNNIGFGDEEDYSDTLTQTLKEWFEVSKKHYPNVLVHHNEVGNTPPPTMSLISTFNENMLRKYMRTAKPDFITYDMYYFRENRQSSEVGGTVIPFYDDLNRYRKVASEGYDGSGLSPIPFGTYLQGWRTGPGAATYEKRGDGWYEITESQAYLSAFANWTFGAKWLSMFRWIEDTPGYLFSDYRPDEDGNWPKYHIYGQYKEMFRQSKNLGEHLIRINNKDVVIVPGQHMKDGQITKNNRPKDNPEWTKSGDRAFIDSLEISNLGKTNHSLKGDVFIGYFDPLPGIDTTQFFTSTAPKYFMLLNGLTSGQGLPAEEQTGSSYETRQEIKVTFDLSGGQARADQLRKVSRLTGELVAAPLKDLGNGKYEMTVVLGGGMADLYFWELGSLNTGNSKPVVADTPHDVRLTGDPKYAKNREIRDLTGKTVTVGWIKDTYSPVPQPLIHYNFSFTKDQNGKLQPMKNPDILSYFTRYYENTLWNKRVERIQKESNVKLEFVADIAWTKQELMDNIRKVKEGQTVDGMPDILIVPDEWTWSGLIQNEMILPASSFSEFDFTERKWNKSYKAMTTWKDQIYGMYAGPTMNSTGLFVNKALQASIGVTDDLMALQQNNAWDWNKLREVASAFQASANREGKYLLAGTDELFKQMVYANGAARGSVGGAMNQEFDLTSSSFREAAELYSELHAAGLIAAKPEGATDDWYVEQFSKNNILFLALPYQQTVDKLKFSYTNQDAVIEMKEGSFLGQPALIPTIVDAYETAYPDGIYKMAQGDWVFLMFPKGPSATGYAAMIDNPAYPVLLSSSANPADAAYVWNILSHEFEGVAYDRFLKLYLNQREVDKTTLKRIGLKEGVWDSYSGTGAWEQVIKPGVLPMLQAGVIDEAKLAELSVEAASYVTNNMTKPAQPG

SEQ ID NO: 5 - protein, 1254 aa, Artificial Sequence
  Description of Artificial Sequence Synthetic polypeptide
SIRQDPTTGNYYKNVPLVGADFDDASNSNIVAKGTWDDNTAPLNTFQNDPAPGQPEQLLLKDGDFEAGAASVTTDTNVQNQFFSKNNQYEIVTGDTASGQYALKLRSPETIGYHKTDLKPSTKYQISFMAKVGSASQKLSFRISGYKNDNPYDLDNVMNYIEHTQMKNTGWSRFYYDLETGPSATSAFIDFSTAAGSTAWIDDVKLVEQGPADPPVTEPTLSRGSRLFLEKGLQIQSWVPTDVAYATRKWMKPPTAEEIVDLGLTTVQYNDAPNYSKTLHEEYKKLQQTNPSLPDLKWGVAFGPNANHLSSSYFDSETIAKHDPNKTGAPTEEQKARGFLTPDQLANVQNLNNIGFGDEEDYSDTLTQTLKEWFEVSKKHYPNVLVHHNEVGNTPPPTMSLISTFNENMLRKYMRTAKPDFITYDMYYFRENRQSSEVGGTVIPFYDDLNRYRKVASEGYDGSGLSPIPFGTYLQGWRTGPGAATYEKRGDGWYEITESQAYLSAFANWTFGAKWLSMFRWIEDTPGYLFSDYRPDEDGNWPKYHIYGQYKEMFRQSKNLGEHLIRINNKDVVIVPGQHMKDGQITKNNRPKDNPEWTKSGDRAFIDSLEISNLGKTNHSLKGDVFIGYFDPLPGIDTTQFFTSTAPKYFMLLNGLTSGQGLPAEEQTGSSYETRQEIKVTFDLSGGQARADQLRKVSRLTGELVAAPLKDLGNGKYEMTVVLGGGMADLYFWELGSLNTGNSKPVVADTPHDVRLTGDPKYAKNREIRDLTGKTVTVGWIKDTYSPVPQPLIHYNFSFTKDQNGKLQPMKNPDILSYFTRYYENTLWNKRVERIQKESNVKLEFVADIAWTKQELMDNIRKVKEGQTVDGMPDILIVPDEWTWSGLIQNEMILPASSFSEFDFTERKWNKSYKAMTTWKDQIYGMYAGPTMNSTGLFVNKALQASIGVTDDLMALQQNNAWDWNKLREVASAFQASANREGKYLLAGTDELFKQMVYANGAARGSVGGAMNQEFDLTSSSFREAAELYSELHAAGLIAAKPEGATDDWYVEQFSKNNILFLALPYQQTVDKLKFSYTNQDAVIEMKEGSFLGQPALIPTIVDAYETAYPDGIYKMAQGDWVFLMFPKGPSATGYAAMIDNPAYPVLLSSSANPADAAYVWNILSHEFEGVAYDRFLKLYLNQREVDKTTLKRIGLKEGVWDSYSGTGAWEQVIKPGVLPMLQAGVIDEAKLAELSVEAASYVTNNMTKPAQPG

(19) United States
(12) Patent Application Publication
(10) Pub. No.: US 2020/0206323 A1
(43) Pub. Date: Jul. 2, 2020

(54) AN AGENT, A DEVICE AND A BLOOD-CIRCULATION SYSTEM FOR TREATING LYSOSOMAL STORAGE DISEASES, AND A METHOD FOR TREATING LYSOSOMAL STORAGE DISEASES

(71) Applicants: THE NEMOURS FOUNDATION, Jacksonville, FL (US); SEIKAGAKU CORPORATION, Tokyo (JP)

(72) Inventors: Shunji TOMATSU, Wilmington, DE (US); Kazuki SAWAMOTO, Tokyo (JP); Takehiko NAKAMURA, Tokyo (JP); Ikue KITAZAWA, Tokyo (JP); Hideyuki FUTATSUMORI, Tokyo (JP)

(73) Assignees: THE NEMOURS FOUNDATION, Jacksonville, FL (US); SEIKAGAKU CORPORATION, Tokyo (JP)

(21) Appl. No.: 16/643,713

(22) PCT Filed: Sep. 7, 2018
(86) PCT No.: PCT/US2018/050057

§ 371 Date: Mar. 2, 2020

Related U.S. Application Data
(60) Provisional application No. 62/556,076, filed on Sep. 8, 2017.
(60) Provisional application No. 62/556,644, filed on Sep. 11, 2017.
(60) Provisional application No. 62/617,940, filed on Jan. 16, 2018.

(51) Int. Cl. A61K 38/47; A61K 38/50; A61K 38/51; A61K 35/14; A61M 1/36

(52) U.S. Cl. CPC A61K 38/47; C12Y 302/01096; A61K 38/50; A61M 1/3687; A61K 38/51; C12Y 402/02008; A61K 35/14; C12Y 302/01103

(57) ABSTRACT

A therapeutic agent containing, as an effective component, a glycolytic enzyme which is different from a deficient protein of a patient with lysosomal storage disease as a subject and/or a glycolytic enzyme which does not have a mannose 6-phosphate moiety or a mannose moiety.

### SEQUENCE LISTING

The instant application contains a Sequence Listing which has been filed electronically in ASCII format and is hereby incorporated by reference in its entirety. Said ASCII copy, created on Aug. 27, 2018, is named 116227-0109_SL.txt and is 65,245 bytes in size.

### TECHNICAL FIELD

The present invention relates to a treatment of lysosomal storage disease using a glycolytic enzyme.

### BACKGROUND ART

In a lysosome as a cell organelle, various kinds of enzymes are present and they are responsible for degradation of various saccharide moieties such as mucopolysaccharides and glycoconjugates in a living body. Most of the lysosomal enzymes present in a living body of mammals including human are exo-type enzymes which cleave a substrate at each constituting unit from the end. In most cases, decomposition of saccharide moieties is achieved based on a stepwise and series of exo-type enzyme reactions.

The lysosomal storage disease is a genetic disease which is caused by abnormality of lysosomal enzymes. An onset of the lysosomal storage disease is supposed to be caused by lowered metabolic activity of lysosomal enzymes, for example, due to enzyme deficiency, malfunction, abnormality in activation mechanism, or the like, which can lead to accumulation of substrates for these enzymes. Among the lysosomal storage diseases, mucopolysaccharidosis, sphingolipidosis, glycogen storage disease type II, glycoprotein storage disease, or the like are known as diseases in which saccharide such as mucopolysaccharide or glycoconjugate accumulates in the lysosome.

As a method for treating lysosomal storage disease, an enzyme replacement therapy (ERT) is known. In ERT, the lowered metabolic activity in lysosome of patient is replenished by administering an enzyme which corresponds to the deficient enzyme in the patient (Patent Literature 1 and Non Patent Literature 1). In a living body, inside of lysosome is maintained at acidic condition with pH of 5 or lower. The deficient enzyme, which is replenished in ERT, is also optimally activated in acidic condition. Due to these reasons, delivery of the enzyme to inside of lysosome, wherein pH is optimum for the replenished enzyme, has been conventionally believed to be important in ERT.

During the process of synthesizing lysosomal enzyme in a living body of mammals, a lysosomal enzyme precursor having an asparagine residue with a high mannose type sugar chain (i.e., N-glycosylated enzyme precursor) is modified with mannose-6-phosphate according to an action of N-acetylglucosamine-1-phosphotransferase. The enzyme modified with mannose-6-phosphate is associated with a mannose-6-phosphate receptor in a golgi body and transported to lysosome (Non Patent Literature 2). Namely, the mannose-6-phosphate moiety of the enzyme is a tag for delivering the enzyme to lysosome. According to ERT for Gaucher disease, one kind of lysosomal storage disease, β-glucocerebrosidase having a mannose moiety is used. Modification of the β-glucocerebrosidase with mannose is also contemplated for the transfer to lysosome. Namely, it is essential for the conventional ERT to promote transferring the deficient enzyme in the patient to lysosome by modification with mannose-6-phosphate or mannose so that the enzyme is allowed to act in inside of lysosome (Patent Literature 1 and Non Patent Literature 1). The mannose-6-phosphate or mannose modified enzyme used for ERT is conventionally produced by expressing the enzyme in mammalian cells or by chemically incorporating the mannose-6-phosphate moiety or mannose moiety to the enzyme.

### CITATION LIST

Patent Literature 1: WO 2012/012718 A
Non Patent Literature 1: Annual Review of Genomics and Human Genetics 2012, 13: 307-35
Non Patent Literature 2: Journal of Biological Chemistry 1989, 264 (21) 12115-12118

### SUMMARY OF INVENTION

According to present invention, an agent, a device, and a blood circulation system for treating lysosomal storage disease using a glycolytic enzyme, a method for producing an agent for treating lysosomal storage disease, and a method for treating lysosomal storage disease are provided.

It has been considered that the most important point for ERT to re-activate the lowered metabolism in lysosome by efficiently delivering the deficient enzyme to lysosome and clearance of the accumulated saccharide moieties there in order to improve the symptom of lysosomal storage disease.

On the other hand, the present invention is at least partially based on a surprising finding that even a treatment for reducing accumulated saccharides present in patient's blood circulation can exhibit an improved or even excellent therapeutic effect on lysosomal storage disease. Namely, the present invention relates to a therapy for lysosomal storage disease by which blood of a patient containing accumulated saccharide moieties is contacted with a glycolytic enzyme so as to reduce the saccharide content in blood.

According to one embodiment, a glycolytic enzyme which is not identical with a deficient protein (for example, a deficient enzyme such as a genetically deficient enzyme or partially inactive enzyme) in a patient with lysosomal storage disease is administered to the patient or contacted with blood of the patient. Namely, unlike the common ERT, the glycolytic enzyme, as long as it has an activity of degrading a saccharide accumulated in lysosome of the patient, is not required to be identical with the deficient enzyme in the patient with lysosomal storage disease.

According to another embodiment, a glycolytic enzyme having no mannose 6-phosphate moiety or mannose moiety is administered to a patient or contacted with the blood of the patient. Namely, the glycolytic enzyme can exhibit an improved or even excellent therapeutic effect for lysosomal storage disease while it is not required also to be delivered to lysosome.

According to some embodiments, a glycolytic enzyme which has different manner of degradation from the original deficient protein in the patient with lysosomal storage disease is administered to the patient or contacted with blood of the patient.

According to some embodiments, a glycolytic enzyme derived from a microorganism having an activity to degrade the accumulated saccharide moieties in the patient with lysosomal storage disease is administered to the patient or contacted with blood of the patient. In a preferred embodiment, a glycolytic enzyme derived from a microorganism with lowered endotoxin level is used.

According to some embodiments, an endo-type enzyme is used as the glycolytic enzyme for the patient with lysosomal storage disease which is selected from the group consisting of mucopolysaccharidosis, sphingolipidosis, glycogen storage disease type II, and glycoprotein storage disease. According to a preferred embodiment, a glycosaminoglycan degrading enzyme is used as the glycolytic enzyme for the patient with mucopolysaccharidosis (for example, Hurler disease, Scheie disease, Hunter disease, Sanfilippo disease (A, B, C or D), Morquio disease (A or B), Maroteaux-Lamy disease, Sly disease, mucopolysaccharidosis type IX, or mucopolysaccharidosis-plus syndrome).

Also provided are a method for producing an agent for treating lysosomal storage disease containing glycolytic enzyme as an effective component, a therapeutic device and a blood circulation system using glycolytic enzyme, and a method for treating lysosomal storage disease.

### BRIEF DESCRIPTION OF DRAWINGS

FIG. 1 is an exemplary diagram illustrating the blood circulation system for treatment and therapeutic method that are related to one aspect of the present invention.

FIG. 2A shows serum levels of keratan sulfate mono-sulfated disaccharide in 8-weeks-old GALNS (N-acetylgalactosamine-6-sulfate sulfatase, referred to also as GALNS here in below) knockout mice after single intravenous administration of PBS (No-treatment group) or keratanase (Treatment group) (mean value±standard deviation).

FIG. 2B shows serum levels of heparan sulfate non-sulfated disaccharide in 8-weeks-old GALNS knockout mice after single intravenous administration of PBS (No-treatment group) or keratanase (Treatment group) (mean value±standard deviation).

FIG. 3A shows the serum levels of keratan sulfate mono-sulfated disaccharide in GALNS knockout mice after repeated intravenous administration of PBS (No-treatment group) or keratanase (Treatment group). PBS or keratanase was administered at 1 day or 2 days after birth, and 4 and 8-weeks of age (mean value±standard deviation).

FIG. 3B shows the serum levels of heparan sulfate non-sulfated disaccharide in GALNS knockout mice after repeated intravenous administration of PBS (No-treatment group) or keratanase (Treatment group). PBS or keratanase was administered at 1 day or 2 days after birth, and 4 and 8-weeks of age (mean value±standard deviation).

FIG. 4A is a representative photomicrograph of the epiphyseal plate of femur from a 12-weeks-old mouse following repeated intravenous administration of PBS (Control group). Hematoxylin and eosin (H&E) stained paraffin section.

FIG. 4B is a representative photomicrograph of the epiphyseal plate of femur from a 12-weeks-old mouse following repeated intravenous administration of PBS (No-treatment group). H&E stained paraffin section.

FIG. 4C is a representative photomicrograph of the epiphyseal plate of femur from a 12-weeks-old mouse following repeated intravenous administration of keratanase (Treatment group). H&E stained paraffin section.

FIG. 5A is a representative photomicrograph of the epiphyseal plate of femur from a 12-weeks-old mouse following repeated intravenous administration of PBS (Control group). Toluidine blue stained resin section.

FIG. 5B is a representative photomicrograph of the epiphyseal plate of femur from a 12-weeks old mouse following repeated intravenous administration of PBS (No-treatment). Toluidine blue stained resin section.

FIG. 5C is a representative photomicrograph of the epiphyseal plate of femur from a 12-weeks old mouse following repeated intravenous administration of keratanase (Treatment group). Toluidine blue stained resin section.

FIG. 6 shows serum levels of keratan sulfate mono-sulfated disaccharide in 4-weeks-old GALNS knockout mice after single intravenous administration of PBS (No-treatment) or keratanase (Treatment) (mean value±standard deviation, n=9, *p<0.05 unpaired t test). N.D.: not detect.

FIG. 7A shows keratan sulfate mono-sulfated disaccharide level in liver of PBS (No-treatment) or keratanse (Treatment) injected GALNS knockout mice (mean value±standard deviation, n=4, *: p<0.05 unpaired t test).

FIG. 7B shows keratan sulfate mono-sulfated disaccharide level in lung of PBS (No-treatment) or keratanse (Treatment) injected GALNS knockout mice (mean value±standard deviation, n=4).

FIG. 7C shows keratan sulfate mono-sulfated disaccharide level in spleen of PBS (No-treatment) or keratanse (Treatment) injected GALNS knockout mice (mean value±standard deviation, n=4, *: p<0.05 unpaired t test).

FIG. 7D shows keratan sulfate mono-sulfated disaccharide level in heart of PBS (No-treatment) or keratanse (Treatment) injected GALNS knockout mice (mean value±standard deviation, n=4).

### DESCRIPTION OF REPRESENTATIVE EMBODIMENTS

Representative embodiments of the present invention are explained hereinbelow, but the present invention is not limited thereto.

As used herein, the terms “a” or “an” shall mean one or more than one.

According to the present invention, an improved or even excellent therapeutic effect for lysosomal storage disease can be achieved. Examples of the therapeutic effect according to the present invention can include a significant improvement of hypochondroplasia in a joint at growth stage. According to the present invention, delivery of the glycolytic enzyme to lysosome in living body of the patient is not required. Thus, this therapy will be effective even for a patient such as who has loss or malfunction of lysosomal trafficking activity caused by a deficiency of mannose-6-phosphate receptors. Furthermore, the present invention can be applicable even to a subject in which the saccharide accumulates in cerebral lysosomes, since it is not necessary to deliver the glycolytic enzyme itself to a brain. Thus, invasive regimen such as intrathecal or intraventricular administration is not required even in such a case.

As used herein, the term “deficient protein” refers to a protein, due to missing or malfunction thereof, ascribed as a cause of accumulation of a substrate in lysosome of lysosomal storage disease patient.

As used herein, the term “deficient enzyme” refers to an enzyme, due to missing or malfunction thereof, ascribed as a cause of accumulation of a substrate in lysosome of lysosomal storage disease patient.

As used herein, the terms “saccharide” and “saccharide moiety”, which are used interchangeably, include a hydrocarbon, a glycosaminoglycan, and a glycoconjugate such as a glycolipid and a glycoprotein.

Examples of the lysosomal storage disease include, although not particularly limited, mucopolysaccharidosis (for example, Hurler disease, Scheie disease, Hunter disease, Sanfilippo disease (A, B, C or D), Morquio disease (A or B), Maroteaux-Lamy disease, Sly disease, mucopolysaccharidosis type IX, or mucopolysaccharidosis-plus syndrome), sphingolipidosis (for example, GM1 gangliosidosis, GM2 gangliosidosis, Fabry disease, Faber disease, Gaucher disease, Niemann-Pick disease, and Krabbe disease), glycogen storage disease type II (Pompe disease), and glycoprotein storage disease (for example, mannosidosis, fucosidosis, and galactosialidosis). According to a preferred embodiment, the lysosomal storage disease is selected from the group consisting of Hurler disease, Scheie disease, Hunter disease, Sanfilippo disease (A, B, C and D), Morquio disease (A and B), Maroteaux-Lamy disease, Sly disease, mucopolysaccharidosis type IX, and mucopolysaccharidosis-plus syndrome.

According to a particularly preferred embodiment, the lysosomal storage disease is Morquio disease (mucopolysaccharidosis IV). Morquio disease types A and B are known to be caused by deficiency of GALNS and β-galactosidase, respectively. The keratan sulfate (formed of β-(1→3) repeat of galactosyl β-(1→4)-N-acetylglucosamine disaccharide unit), which is accumulated in a patient with Morquio disease, is degraded from the non-reducing end in a healthy person by the following (1) and (2) repeated reaction.

(1) Desulfation of the 6-O-sulfate group from the non-reducing terminal saccharide residue by N-acetylgalactosamine-6-sulfatase (referred to also as GALNS hereinbelow) or N-acetylglucosamine-6-sulfatase

(2) Hydrolysis reaction for eliminating non-reducing terminal saccharide residue by β-galactosidase (GLB1) or β-hexosaminidase (HexA)

The glycolytic enzyme can be endoenzyme or exoenzyme. According to a preferred embodiment, the glycolytic enzyme is an endoenzyme.

From the viewpoint of the glycolytic activity in a neutral pH condition, a glycolytic enzyme derived from microorganisms is used according to a preferred embodiment. Non-limiting examples of the microorganisms include microorganisms belonging to the genera Bacillus, Escherichia, Pseudomonas, Flavobacterium, Proteus, Arthrobacter, Streptococcus, Bacteroides, Aspergillus, Elizabethkingia, and Streptomyces. In particular, from the viewpoint of high glycolytic activity, a glycolytic enzyme derived from a microorganism of genus Bacillus is used in a more preferred embodiment.

Examples of the glycolytic enzyme which can be used in the present invention include, although not particularly limited, a glycosaminoglycan degrading enzyme, a glycosidase, and a peptide:N-glycanase (PNGase).

According to a preferred embodiment, at least one selected from a group consisting of a glycosaminoglycan degrading enzyme (for example, keratanase such as keratanase I or keratanase II; heparinase such as heparinase I, heparinase II, or heparinase III; heparitinase such as heparitinase IV, heparitinase V, heparitinase T-I, heparitinase T-II, heparitinase T-III, or heparitinase T-IV; chondroitinase such as chondroitinase ABC, chondroitinase AC, chondroitinase ACIII, chondroitinase B, or chondroitinase C; hyaluronidase such as hyaluronidase derived from Actinomycetes or hyaluronidase derived from Streptococcus); glucosidase (for example, β-galactosidase or α-galactosidase derived from microorganisms); and a peptide:N-glycanase (PNGaseF, endoglycosidase H, or the like) is used as a glycolytic enzyme.

According to one embodiment, at least one selected from a group consisting of keratanase, heparinase, heparitinase, chondroitinase, hyaluronidase, β-galactosidase, α-galactosidase, PNGaseF, and endoglycosidase H is used as a glycolytic enzyme.

According to a more preferred embodiment, at least one selected from a group consisting of keratanase, heparinase, heparitinase, chondroitinase, and hyaluronidase is used as a glycolytic enzyme.

Examples of keratanase include, but not limited thereto, keratanase derived from microorganisms of genus Bacillus including endo-β-N-acetylglucosaminidase derived from Bacillus sp. Ks36 (Hashimoto Shinichi, Morikawa Kiyoshi, Kikuchi Hiroshi, Yoshida Keiichi, and Tokuyasu Kiyochika, Biochemistry, 60, 935 (1988)) and endo-β-N-acetylglucosaminidase derived from Bacillus circulans KsT202 (Clinical Biochemistry 48 (2015) 796-802); keratanase derived from microorganisms of genus Escherichia including endo-β-galactosidase derived from Escherichia freundii (H. Nakagawa, T. Yamada, J-L. Chien, A. Gardas, M. Kitamikado, S-C. Li, Y-T. Li, J. Biol. Chem., 255, 5955 (1980)); and keratanase derived from microorganisms of genus Pseudomonas including endo-β-galactosidase derived from Pseudomonas sp. IFO-13309 strain (K. Nakazawa, N. Suzuki, S. Suzuki, J. Biol. Chem., 250, 905 (1975), K. Nakazawa, S. Suzuki, J. Biol. Chem., 250, 912 (1975)) and endo-β-galactosidase produced by Pseudomonas reptilivora (JP 57-41236 B).

Examples of the heparinase include, but not limited thereto, heparinase derived from microorganisms of genus Flavobacterium such as Flavobacterium heparinum (U.S. Pat. No. 4,443,545); heparinase derived from microorganisms of genus Bacillus such as Bacillus sp BH100; and heparinase derived from microorganisms of genus Bacteroides such as heparinase I, heparinase II or heparinase III that are derived from Bacteroides Eggerthii (Glycobiology., 21, 1454-1531 (2011)).

Examples of the heparitinase include, but not limited thereto, heparitinase derived from microorganisms of genus Flavobacterium such as heparitinase IV or heparitinase V that are derived from Flavobacterium sp. Hp206 (JP 02-057183 A); and heparitinase derived from microorganisms of genus Bacillus such as heparitinase T-I, heparitinase T-II, heparitinase T-III or heparitinase T-IV that are derived from Bacillus circulans HpT298 (U.S. Pat. No. 5,290,695).

Examples of the chondroitinase include, but not limited thereto, chondroitinase derived from microorganisms of genus Proteus such as chondroitinase ABC derived from Proteus vulgaris (T. Yamagata, H. Saito, O. Habuchi, S. Suzuki, J. Biol. Chem., 243, 1523 (1968), S. Suzuki, H. Saito, T. Yamagata, K. Anno, N. Seno, Y. Kawai, T. Furuhashi, J. Biol. Chem., 243, 1543 (1968)); and chondroitinase derived from microorganisms of genus Flavobactericum such as chondroitinase AC derived from Flavobacterium heparinum (T. Yamagata, H. Saito, O. Habuchi, S. Suzuki, J. Biol. Chem., 243, 1523 (1968)), chondroitinase ACIII derived from Flavobacterium sp. Hp102 (Miyazono Hirofumi, Kikuchi Hiroshi, Yoshida Keiichi, Morikawa Kiyoshi, and Tokuyasu Kiyochika, Biochemistry, 61, 1023 (1989)), chondroitinase B derived from Flavobacterium heparinum (Y. M. Michelacci, C. P. Dietrich, Biochem. Biophys. Res. Commun., 56, 973 (1974)), or chondroitinase C derived from Flavobacterium sp. Hp102 (Miyazono Hirofumi, Kikuchi Hiroshi, Yoshida Keiichi, Morikawa Kiyoshi, and Tokuyasu Kiyochika, Biochemistry, 61, 1023 (1989)).

Examples of the hyaluronidase include, but not limited thereto, hyaluronidase derived from microorganisms of genus Streptomyces such as Streptomyces hyalurolyticus; and hyaluronidase derived from microorganisms of genus Streptococcus such as Streptococcus pyogenes.

Examples of the β-galactosidase include, but not limited thereto, β-galactosidase derived from microorganisms of genus Aspergillus such as Aspergillus oryzae; β-galactosidase derived from microorganisms of genus Streptococcus such as Streptococcus pneumoniae; and β-galactosidase derived from microorganisms of genus Escherichia such as Escherichia coli.

Examples of the α-galactosidase include, but not limited thereto, α-galactosidase derived from microorganisms of genus Aspergillus such as Aspergillus oryzae; and α-galactosidase derived from microorganisms of genus Escherichia such as Escherichia coli.

Examples of the PNGaseF include, but not limited thereto, PNGaseF derived from microorganisms of genus Elizabethkingia such as Elizabethkingia meningoseptica; and PNGaseF derived from microorganisms of genus Flavobacterium such as Flavobacterium meningosepticum.

Examples of the endoglycosidase H include, but not limited thereto, endoglycosidase H derived from microorganisms of genus Streptomyces such as Streptomyces plicatus.

The microorganisms may be obtained from an institute such as American Type Culture Collection (ATCC) or National Institute of Technology and Evaluation (NITE). A commercially available enzyme preparation may be used as a glycolytic enzyme.

The glycolytic enzyme derived from microorganisms may have the same amino acid sequence with that of the native enzyme present in the microorganism. Further, as long as it has a degrading activity of accumulated saccharide moieties in lysosome in patient, the glycolytic enzyme may have an amino acid sequence which a part of amino acid in the sequence of native enzyme is substituted, added, inserted and/or deleted. Non-limiting examples of the glycolytic enzyme derived from microorganisms include an enzyme containing a partial polypeptide from Ser³⁵ to Gly¹⁵⁰² of keratanase derived from Bacillus circulans KsT202 having an amino acid sequence of SEQ ID:2 (Clinical Biochemistry 48 (2015) 796-802), and a polypeptide chain in which at least one of the domain C (from Phe⁸¹ to Thr¹⁹²) and the domain D (from Ala²²⁷ to Ala²⁹⁴) of said partial polypeptide is additionally deleted from the amino acid sequence of SEQ ID:2 (Glycoconjugate Journal (2017), Volume 34, Issue 5, 643-649; DOI 10.1007/s10719-017-9786-3), for example, a polypeptide having an amino acid sequence of SEQ ID:3, 4 or 5. According to one embodiment of the present invention, a protein having an amino acid sequence with an identity of 80% or higher, preferably 85% or higher, more preferably 90% or higher, even more preferably 95% or higher, particularly preferably 99% or higher to the amino acid sequence any one of SEQ ID NO: 1 to 5 and having the degrading activity of accumulated saccharide moieties in lysosome in the patient is used as the glycolytic enzyme.

According to a preferred embodiment, a glycolytic enzyme produced by microorganisms is used. Compared to a glycolytic enzyme produced using animal cells as host cells, the glycolytic enzyme produced using microorganisms as host cells can be provided as an enzyme having uniform quality among manufacturing lots at lower cost. Preferred examples of the microorganisms for producing the glycolytic enzyme include microorganisms belonging to the genera Bacillus and Escherichia (for example. E. coli) as described above. The glycolytic enzyme produced by using microorganisms as host cells can be a naturally-occurring enzyme of the host microorganism (i.e., enzyme derived from the microorganism itself) or an enzyme obtained from the genetically engineered microorganism so as to produce a desired enzyme.

According to one embodiment, a glycolytic enzyme which is not substantially transferred to lysosome after administration into a patient is used. The term “not substantially transferred” means that the amount of glycolytic enzyme transferring to lysosome compared to the entire amount of administered glycolytic enzyme is an amount which does not contribute to obtainment of a desired response under the consideration of the descriptions of the present specification and the state of the art.

The glycolytic enzyme may be used either singly or in combination of two or more thereof. In a case where two or more kinds of saccharides are accumulated in the patient, the glycolytic enzyme having an activity of degrading at least one of these saccharides is used. Alternatively, two or more of glycolytic enzymes for each of the accumulated saccharides may be used in combination.

The glycolytic enzyme may be modified with a conventionally known chemical group including, but not limited thereto, an acetyl group, a polyalkylene group (for example, polyethylene glycolation), an alkyl group, an acyl group, a biotin, a label (for example, labeling with fluorescent material, luminescent material, or the like), a phosphate group, and a sulfate group.

According to one aspect, as the glycolytic enzyme, an enzyme that is different from the deficient protein of the patient with a lysosomal storage disease and has a degrading activity of the accumulated saccharide moieties in lysosome in the patient is used. In this embodiment, since the glycolytic enzyme used for the therapy is different from the deficient protein, it can be a therapy which is also effective for a patient having a neutralizing antibody against the enzyme used for ERT.

As used herein, the term “different from a deficient protein” refers to a protein not having a continuous polypeptide chain in which the amino acid sequence identity to the deficient protein of the patient is, for example, 90% or more and 100% or less, 80% or more and 100% or less, 70% or more and 100% or less, 60% or more and 100% or less, or 50% or more and 100% or less. The glycolytic enzyme may have, for example, less than 99%, preferably less than 90%, more preferably less than 85%, even more preferably less than 70%, still even more preferably less than 50%, most preferably less than 30% of an amino acid sequence identity to the deficient protein in the patient.

As used herein, the term “different from a deficient enzyme” refers to an enzyme not having a continuous polypeptide chain in which the amino acid sequence identity to the deficient enzyme of the patient is, for example, 90% or more and 100% or less, 80% or more and 100% or less, 70% or more and 100% or less, 60% or more and 100% or less, or 50% or more and 100% or less. The glycolytic enzyme may has, for example, less than 99%, preferably less than 90%, more preferably less than 85%, even more preferably less than 70%, still even more preferably less than 50%, most preferably less than 30% of an amino acid sequence identity to the deficient enzyme in the patient. The amino acid identity can be evaluated by using Clustal W (Thompson, J. D., Higgins, D. G., and Gibson, T. J. (1994) Nucleic Acids Res. 22, 4673-4680), for example.

According to one embodiment, the degrading enzyme of which cleavage site is not identical with the deficient enzyme in the patient with lysosomal storage disease can be used as the glycolytic enzyme different from the original deficient enzyme. Namely, the enzyme is able to cleavage at a different glycosidic bond from that of the original deficient enzyme in the patient with lysosomal storage disease. For example, keratanase II recognizes N-acetylglucosamine-6-sulfate on the keratan sulfate (KS) and hydrolyzes KS between the 4 GlcNAc β1-3 galactose with an endolytic manner. Thus, the cleavage site of keratanase II is not identical with that of GALNS (a coding gene thereof is a recessive gene in Morquio A patient.) or β-galactosidase (a coding gene thereof is a recessive gene in Morquio B patient).

According to one embodiment, the glycolytic enzyme derived from a different biospecies than the patient with lysosomal storage disease as subject is used as the glycolytic enzyme. According to a preferred embodiment, the patient with lysosomal storage disease is human, and the enzyme derived from microorganism which able to degrade the accumulated saccharide moieties in lysosome in the patient is used as the therapeutic glycolytic enzyme.

The relationship between the deficient protein and accumulated saccharide moiety in lysosome of the patient is already well known for each lysosomal storage disease. Examples of the glycolytic enzyme which is different from the deficient protein and has a degrading activity of accumulated saccharide moieties are listed in the following tables. However, the technical scope of the present invention is not limited thereto.

TABLE 1

| Mucopolysaccharidosis | | | | |
|---|---|---|---|---|
| | | | Accumulated | |
| | Alias | Deficient protein | saccharide(s) | Glycolytic enzyme |
| Mucopolysaccharidosis | Hurler disease, | α-L-Iduronidase | Dermatan sulfate | Dermatan sulfate: chondroitinase |
| Type I | and Scheie | | Heparan sulfate | B, chondroitinase ABC |
| | disease | | | Heparan sulfate: heparinase, |
| | | | | heparitinase |
| Mucopolysaccharidosis | Hunter disease | Iduronate sulfatase | Dermatan | Dermatan sulfate: chondroitinase |
| Type II | | | sulfate, | B, chondroitinase ABC |
| | | | Heparan sulfate | Heparan sulfate: heparinase, |
| | | | | heparitinase |
| Mucopolysaccharidosis | Sanfilippo | Heparan N-sulfatase | Heparan sulfate | Heparan sulfate: heparinase, |
| Type III type A | disease | | | heparitinase |
| Mucopolysaccharidosis | | α-N-Acetylglucosaminidase | | |
| Type III type B | | | | |
| Mucopolysaccharidosis | | Acetyl-CoA: α-glucosaminide | | |
| Type III type C | | acetyltransferase | | |
| Mucopolysaccharidosis | | N-Acetylglucosamin-6-sulfatase | | |
| Type III type D | | | | |
| Mucopolysaccharidosis | Morquio disease | Galactose-6-sulfatase | Keratan sulfate, | Keratan sulfate: keratanase |
| Type IV type A | | | Chondroitin | Chondroitin sulfate: |
| | | | sulfate | chondroitinase ABC, |
| Mucopolysaccharidosis | | β-Galactosidase | Keratan sulfate | chondroitinase AC, chondroitinase |
| Type IV type B | | | | ACIII, chondroitinase C |
| Mucopolysaccharidosis | Maroteaux-Lamy | N-Acetylgalactosamin-4- | Dermatan sulfate | Dermatan sulfate: chondroitinase |
| Type VI | disease | sulfatase | | B, chondroitinase ABC |
| Mucopolysaccharidosis | Sly disease | β-Glucuronicase | Dermatan | Dermatan sulfate: chondroitinase |
| Type VII | | | sulfate, | B, chondroitinase ABC |
| | | | Heparan sulfate | Heparan sulfate: heparinase, |
| | | | | heparitinase |
| Mucopolysaccharidosis | | Hyaluronidase | Hyaluronic acid | Hyaluronic acid: hyaluronidase, |
| Type IX | | | | chondroitinase ABC |
| Mucopolysaccharidosis- | | Vacuolar protein sorting 33 | Heparan sulfate | Heparan sulfate: heparinase, |
| plus syndrome | | homolog A (S. cerevisiae) | | heparitinase |

TABLE 2

| Sphingolipidosis | | | |
|---|---|---|---|
| | Deficient | Accumulated | Glycolytic |
| | protein | saccharide(s) | enzyme |
| GM1 | β- | GM1-Ganglioside, | β-Galactosidase |
| Gangliosidosis | Galactosidase | Asialo GM1- | (derived from |
| | | ganglioside | microorganisms) |

TABLE 3

| Glycogen storage disease type II | | | |
|---|---|---|---|
| | Deficient | Accumulated | Glycolytic |
| | protein | saccharide(s) | enzyme |
| Pompe disease | α-Glucosidase | Glycogen | α-Glycosidase |
| | | | (derived from |
| | | | microorganisms) |

TABLE 4

| Glycoprotein storage disease | | | |
|---|---|---|---|
| | Deficient | Accumulated | Glycolytic |
| | protein | saccharide(s) | enzyme |
| Mannosi- | Mannosidase | Oligosaccharides | Endoglycosidase |
| dosis | | and proteins having | H, PNGaseF |
| | | mannose residue | |
| Fucosidosis | Fucosidase | Oligosaccharides | PNGaseF |
| | | and proteins having | |
| | | fucose residue | |
| Galactosial- | β-galactosidase, | Siallyloligosac- | Endoglycosidase |
| dosis | α-neuraminidase | charides and proteins | H, PNGaseF |

According to one aspect, an enzyme not having a mannose-6-phosphate moiety or a mannose moiety is used as a glycolytic enzyme. Since the glycolytic enzyme not having mannose-6-phosphate moiety or mannose moiety is not necessary to be produced by animal cells, such an enzyme can be produced in a large amount at low cost by using microorganisms such as genus Bacillus, genus E. coli, or the like.

The desired glycolytic enzyme not having mannose-6-phosphate moiety or mannose moiety can be obtained by protein expression system using microorganisms such as genus Bacillus or genus E. coli which does not have an N-glycosylation system (see, Process Biochemistry 47 (2012) 2097-2102). Namely, according to one embodiment of the present invention, the glycolytic enzyme produced from the microorganism not having N-glycosylation system (for example, microorganisms not having UDP-GlcNAc phosphotransferase or an enzyme which has a corresponding catalytic activity of that) is used. According to this embodiment, the desired glycolytic enzyme can be obtained as a naturally-occurring enzyme of the microorganism (i.e., enzyme derived from genus Bacillus) or as a recombinant enzyme from genetically engineered microorganism, for example described hereinbelow.

The above mentioned glycolytic enzymes can be obtained by a known purification process or a known genetic engineering technique. An exemplary method for producing the glycolytic enzyme includes; a step of obtaining culture of host cells selected from a group consisting of a microorganism and animal cell for producing the glycolytic enzyme; and a step of collecting the glycolytic enzyme from the culture. The glycolytic enzyme produced by the host cell may be a naturally-occurring enzyme of the host cell (i.e., enzyme derived from the host cell itself) or an enzyme from the genetically engineered host cell so as to produce the desired enzyme.

One embodiment of the present invention relates to a method for producing a therapeutic agent for lysosomal storage disease which contains a glycolytic enzyme, for example the glycolytic enzyme described above, as an effective component, in which the method includes a step of obtaining a culture of microorganism which produces the glycolytic enzyme, a step of collecting the glycolytic enzyme from the culture, and, optionally, a step of formulating a composition by mixing the collected glycolytic enzyme with a pharmaceutically acceptable additive. Examples of the microorganisms used for producing the glycolytic enzyme include, but not limited thereto, microorganisms belonging to the genera Bacillus, Escherichia, Pseudomonas, Flavobacterium, Proteus, Arthrobacter, Streptococcus, Bacteroides, Aspergillus, Elizabethkingia, or Streptomyces. Preferably, the microorganism is at least one selected from the group consisting of microorganisms of genus Bacillus and microorganisms of genus Escherichia. A skilled person in the art shall be able to determine the culture condition of the microorganisms (i.e., culture medium components, temperature condition, or the like) in accordance with a common method. By producing a glycolytic enzyme using microorganisms, a large amount of the enzyme can be produced at low cost compared to a case where the glycolytic enzyme is produced by using animal cells. The glycolytic enzyme produced by the microorganism may be a naturally-occurring enzyme of the microorganism or an enzyme from the genetically engineered microorganism so as to produce the desired enzyme.

The method for producing glycolytic enzyme may include a step of introducing a recombinant vector for expressing a gene encoding the target glycolytic enzyme to a host.

As for the vector, a suitable vector (for example, phage vector, plasmid vector, or the like) capable of expressing a gene introduced thereto (preferably containing a regulatory sequence like promoter) can be used. The vector is suitably selected in accordance with host cells. More specifically, examples of host-vector system include, but not limited thereto, a combination of E. coli with expression vector for prokaryotic cells like pET series, pTrcHis, pGEX, pTrc99, pKK233-2, pEZZ18, pBAD, pRSET, and pSE420; and a combination of mammalian cells such as COS-7 cells and HEK293 cells with an expression vector for mammalian cells like pCMV series, pME 18S series, and pSVL series; and a combination of host cell selected from a group consisting of insect cells, yeast and Bacillus subtilis, and various vectors corresponding to these cells. From the viewpoint of productivity and production cost, a microorganism (for example, E. coli) is used as the host cell according to a preferred embodiment.

Furthermore, as for the vector, a vector constructed so as to express a protein encoded by an inserted gene or a fusion protein with marker peptide or signal peptide can be also used. Examples of the peptide include a protein A, insulin signal sequence, His tag, FLAG tag, CBP (calmodulin binding protein), and GST (glutathione-S-transferase). The expression vector can be constructed by treating polynucleotide containing targeted sequence and the vector with a restriction enzyme so that the target nucleotide sequence can be inserted into the vector, in accordance with a common method.

A host cell can be transformed with the expression vector in accordance with a common method. For example, in accordance with a method using a commercially available reagent for transfection or a DEAF-dextrin method, an electroporation method, or a method based on gene gun, the expression vector can be introduced to the host.

A skilled person in the art shall determine the culture condition of the microorganisms or animal cells for producing glycolytic enzyme (i.e., culture medium, culture condition, or the like) in accordance with a common method. When E. coli is used as the host cell, for example, a culture medium can be prepared by using LB medium or the like as a main component. Furthermore, when COS-7 cells are used as the host cell, the cells can be cultured at 37° C. by using DMEM containing about 2% (v/v) of fetal bovine serum.

The glycolytic enzyme from a cultured product can be collected by a known method for extraction and purification of proteins depending on the glycolytic enzyme to be produced. For example, if the glycolytic enzyme is produced in soluble form secreted to a culture medium (i.e., supernatant of culture medium), it is possible that the culture medium is recovered and used as a glycolytic enzyme as it is, if required. Furthermore, if the glycolytic enzyme is produced in soluble form that is secreted in cytoplasm or in insoluble form (i.e., membrane binding form), the glycolytic enzyme may be extracted, for example, according to using a nitrogen cavitation device, homogenization, a glass beads mill method, a sonication method, an osmotic shock method, a freezing and thawing method, extraction using surfactant, or a combination thereof. The glycolytic enzyme can be purified by a conventionally known means like salting out, ammonium sulfate fractionation, centrifuge, dialysis, ultrafiltration, adsorption chromatography, ion exchange chromatography, hydrophobic chromatography, reverse phase chromatography, gel permeation chromatography, affinity chromatography, electrophoresis, and a combination thereof.

In one embodiment, the method for producing the glycolytic enzyme includes a step of lowering endotoxin level in the collected glycolytic enzyme to the extent that the endotoxin is substantially not contained. The expression “endotoxin is substantially not contained” means that concentration of the endotoxin is not more than medically acceptable level so that the resultant enzyme is suitable as a material used for treatment of lysosomal storage disease. In one embodiment, the endotoxin level in the collected glycolytic enzyme may be lowered by chromatographic purification. In one embodiment, the endotoxin level is lowered to less than or equal to 50 Endotoxin Unit/mg protein. In a preferred embodiment, the endotoxin level is lowered to less than or equal to 15 Endotoxin Unit/mg protein. The endotoxin level can be measured according to a method described in the Japanese Pharmacopoeia 16th edition, for example by using ENDOSPECY™ (ES-50M, manufactured by Seikagaku Corporation Bio Business).

The recovered glycolytic enzyme may be dried by a known method like freeze drying, if required.

As used herein, the term “treatment” includes not only a complete cure but also improvement or amelioration in symptom of the disease, suppression of the disease progress (including maintaining and lowering the progress rate), and prevention of the disease. Herein, the prevention includes, although not limited thereto, preventing in advance various symptoms of a disorder when those various symptoms accompanying a disorder like low body height, delayed mental development, movement disorder, hepatosplenomegaly, peculiar faces, bone abnormality, imperfect joint formation, joint stiffness, limb pain, joint pain, sweating disorder, macroglossia, hearing loss, respiratory disease, and/or neuronal disorder have not occurred yet. Furthermore, the prevention includes, in case of having an onset of various symptoms accompanying a disorder like body and mental functional disorder although a clear organic lesion has not been recognized, preventing in advance an onset of the organic lesion and suppressing the progress of a symptom that is not exhibited yet among those various symptoms, for example.

As used herein, the term “as an effective component” indicates that the amount of the component is suitable for reasonable risk/benefit ratio and sufficient for obtaining a desired outcome without excessive adverse effects (toxicity, stimulation, or the like). The effective amount may vary depending on various factors including a symptom, a build, age, sex, or the like of a patient to be a subject for administration. However, a person skilled in the art would be able to determine the effective amount with reference to the specific examples as described hereinafter and common technical knowledge.

As used herein, the “patient” refers to an animal, preferably a mammal (for example, human, mouse, rat, hamster, marmot, rabbit, dog, cat, and horse), and more preferably a human.

One aspect of the present invention relates to a therapeutic agent for lysosomal storage disease which contains the glycolytic enzyme described above as an effective component.

A further aspect of the present invention relates to a therapeutic method for lysosomal storage disease which includes administering the above therapeutic agent to a patient in need thereof.

The dosage form and administration route for administering, either in vivo or in vitro, the therapeutic agent can be suitably selected according to the disease to be treated or severity thereof. For example, the glycolytic enzyme can be administered, either parenterally or orally, as it is or as a pharmaceutical composition with other additive(s) such as pharmaceutically acceptable carrier, vehicle, and diluent (for example, as an injectable, a tablet, a capsule, a liquid preparation, a ointment, or a gel preparation). According to a preferred embodiment, the therapeutic agent is formulated into an injectable (for example, intravenous injection, subcutaneous injection, intraspinal injection, intramuscular injection, hypodermic injection, intraperitoneal injection, and intraarticular injection).

The blending amount and dosage of the glycolytic enzyme as an effective component of the therapeutic agent are individually determined corresponding to administration method, administration form, purpose of use of the preparation, specific symptom of the patient, body weight of the patient, or the like. The blending amount and dosage of the glycolytic enzyme can be, in terms of clinical dosage, about 10 ng/kg to 100 mg/kg per day, although it is not particularly limited thereto. The agent may be administered singly or repeatedly to the patient. Furthermore, when repeatedly administered, the interval of administration may be varied between 1 to 10 weeks.

According to the present invention, the glycolytic enzyme is not necessary to be delivered to lysosome in living body of the patient. Actually, according to some aspects of the present invention, the glycolytic enzyme may be used so as to be contacted with blood from the patient, rather than administered to the patient, for the purpose of treating lysosomal storage disease.

According to one aspect of the present invention, a device for treating lysosomal storage disease which includes a carrier and the glycolytic enzyme immobilized to the carrier is provided.

The carrier is not particularly limited as long as it can immobilize the glycolytic enzyme and is insoluble in water and blood. The shape of the carrier is not particularly limited, including a microparticle, a bead, a plate (for example, microplate well), a tube, and a membrane (for example, filter form, hollow fiber form, and flat membrane form). Examples of a material of the carrier include, but not limited thereto, agarose, cellulose, cellulose ester, polystyrene, polypropylene, polyvinyl chloride, nitrocellulose, nylon, polyacrylamide, polyethylene, polypropylene, polyamide, and silica.

The glycolytic enzyme can be immobilized by binding either physically or chemically the glycolytic enzyme to the carrier using a general method such as physical adsorption, covalent binding, or entrapping (see, Immobilized Enzyme, 1975, published by Kodansha, pages 9 to 75).

The embodiment of the therapeutic device is not particularly limited as long as saccharides accumulated in lysosome of the patient can be degraded by contacting blood of the patient with the glycolytic enzyme. Examples of the shape of the therapeutic device include, but not limited thereto, a microparticle, a bead, a plate (for example, microplate well), a tube, and a membrane (filter form, hollow fiber form, and flat membrane form). According to some embodiments, the therapeutic device consists of a column immobilized with the glycolytic enzyme, for example with microparticulate enzymes. Examples of a material of the column include, but not limited thereto, polycarbonate, polystyrene, ABS resin, and glass.

As used herein, the term “blood” shall mean an unprocessed and a processed blood, including a whole blood, a serum, a plasma, and a blood component.

One aspect of the present invention is a blood circulation system provided with the above therapeutic device, a blood sampling-side circuit for transporting blood taken from a patient with lysosomal storage disease to the device, a blood reinfusing-side circuit for transporting the blood contacted with a glycolytic enzyme contained in the therapeutic device to the patient with lysosomal storage disease, and a blood pump for pumping the blood through the blood sampling-side circuit and the blood reinfusing-side circuit. The blood circulation system may be equipped with two or more blood pumps. One embodiment of the present invention relates to a method for treating lysosomal storage disease using the blood circulation system.

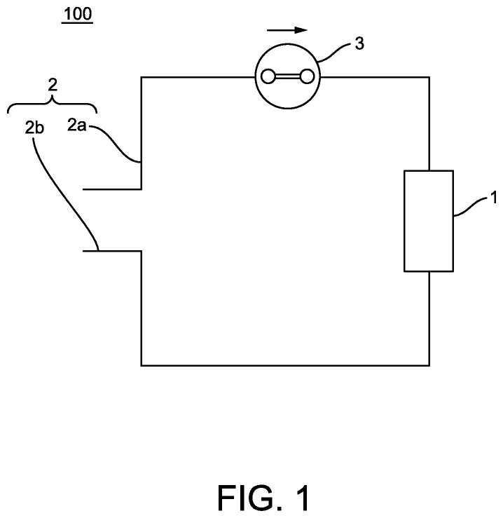

One aspect of the present invention is a method for treating lysosomal storage disease including a step of contacting blood taken from a patient with lysosomal storage disease with the glycolytic enzyme and a step of transporting the blood contacted with the glycolytic enzyme to the patient. FIG. 1 is a schematic diagram illustrating blood circulation system (100) for treating lysosomal storage disease. However, the embodiments of the present invention are not limited to the mode of FIG. 1.

According to the therapeutic blood circulation system (100), blood of the patient is supplied, with an aid of a blood pump (3), to the therapeutic device (1) via the blood sampling-side circuit (2a). With regard to the therapeutic device (1), blood taken from the patient with lysosomal storage disease is brought into contact with the glycolytic enzyme contained in the therapeutic device (1), and thereby elevated saccharides in the blood are allowed to be degraded at the contact with the glycolytic enzyme. The blood brought into contact with the glycolytic enzyme is transported, with an aid of a blood pump (3), to a patient with lysosomal storage disease via a blood-reinfusing side circuit (2b). On a blood circuit (2), an arterial pressure meter, a venous pressure meter, and/or an inlet for administering pharmaceuticals or the like that are not illustrated may be provided.

For the treatment of lysosomal storage disease, whole blood or blood plasma of the patient may be used.

The therapeutic device may be either implantable or extracorporeal circulation equipment.

### EMBODIMENTS

Hereinbelow, preferred embodiments of the present invention are illustrated, but the present invention is not limited thereto;

(1) A therapeutic agent for lysosomal storage disease including a glycolytic enzyme as an effective component, wherein

the glycolytic enzyme is different from a deficient protein of a patient with lysosomal storage disease as a subject and has an activity of degrading a saccharide accumulated in lysosome of the patient.

(2) The therapeutic agent according to (1) in which the glycolytic enzyme does not have a mannose-6-phosphate moiety or a mannose moiety.
(3) The therapeutic agent according to (1) or (2) in which the glycolytic enzyme is different from the deficient protein of the patient with lysosomal storage disease in terms of a cleavage site on the saccharide accumulated in lysosome of the patient.
(4) A therapeutic agent for lysosomal storage disease including a glycolytic enzyme as an effective component in which the glycolytic enzyme does not have a mannose-6-phosphate moiety or a mannose moiety.
(5) The therapeutic agent according to any one of (1) to (4) in which the glycolytic enzyme is an endo-type enzyme.
(6) The therapeutic agent according to any one of (1) to (5) in which the lysosomal storage disease is selected from a group consisting of mucopolysaccharidosis, sphingolipidosis, glycogen storage disease type II, and glycoprotein storage disease.
(7) The therapeutic agent according to any one of (1) to (6) in which the lysosomal storage disease is selected from a group consisting of Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, and mucopolysaccharidosis-plus syndrome.
(8) The therapeutic agent according to any one of (1) to (7) in which the glycolytic enzyme is selected from a group consisting of glycosaminoglycan degrading enzyme, glycosidase, and peptide:N-glycanase.
(9) The therapeutic agent according to any one of (1) to (8) in which the glycolytic enzyme is at least one selected from a group consisting of keratanase, heparinase, heparitinase, chondroitinase, hyaluronidase, β-galactosidase, α-galactosidase, PNGaseF, and endoglycosidase H.
(10) The therapeutic agent according to any one of (1) to (9) in which the glycolytic enzyme is derived from a microorganism.
(11) The therapeutic agent according to (10) in which the microorganism is a microorganism belonging to genus Bacillus.
(12) The therapeutic agent according to any one of (1) to (11) in which the patient is a human.
(13) The therapeutic agent according to any one of (1) to (12) in which a host cell producing the glycolytic enzyme is a microorganism.
(14) The therapeutic agent according to any one of (1) to (13) in which the therapeutic agent is formulated into an injectable preparation.
(15) A method for producing a therapeutic agent for lysosomal storage disease containing a glycolytic enzyme as an effective component, the method including:

a step of obtaining a culture of microorganism which produces the glycolytic enzyme;

a step of collecting the glycolytic enzyme from the culture; and

optionally, a step of formulating a composition by mixing the collected glycolytic enzyme with a pharmaceutically acceptable additive.

(16) The method according to (15), wherein the microorganism is selected from the group consisting of a microorganism belonging to genera Bacillus and Escherichia.
(17) The method according to (15) or (16), wherein the method includes a step of lowering endotoxin level in the collected glycolytic enzyme to the extent that the endotoxin is substantially not contained.
(18) The method according to any one of (15) to (17) in which the therapeutic agent is selected from any one of (1) to (14).
(19) A therapeutic device for lysosomal storage disease including:

a carrier and a glycolytic enzyme immobilized to the carrier, and

the glycolytic enzyme is different from a deficient protein of a patient with lysosomal storage disease as a subject and has an activity of degrading a saccharide accumulated in lysosome of the patient.

(20) A therapeutic device for lysosomal storage disease including:

a carrier and a glycolytic enzyme immobilized to the carrier, and

the glycolytic enzyme does not have a mannose-6-phosphate moiety or a mannose moiety.

(21) A blood circulation system for treating lysosomal storage disease including:

the therapeutic device according to (19) or (20);

a blood sampling-side circuit for transporting blood taken from a patient with lysosomal storage disease to the therapeutic device;

a blood reinfusing-side circuit for transporting the blood contacted with the glycolytic enzyme contained in the therapeutic device to the patient with lysosomal storage disease; and

a blood pump for pumping the blood inside the blood sampling-side circuit and the blood reinfusing-side circuit.

(22) A method for treating lysosomal storage disease including administering the therapeutic agent according to any one of (1) to (14) to a patient in need thereof.
(23) A method for treating lysosomal storage disease including:

a step of contacting blood taken from a patient with lysosomal storage disease with a glycolytic enzyme; and

a step of transporting the blood contacted with the glycolytic enzyme to the patient, and

the glycolytic enzyme is different from a deficient protein of the patient and has an activity of degrading a saccharide accumulated in lysosome of the patient.

(24) The method according to (23), wherein the glycolytic enzyme does not have a mannose-6-phosphate moiety or a mannose moiety.
(25) The method according to (23) or (24), wherein the glycolytic enzyme is different from the deficient protein of the patient with lysosomal storage disease in terms of a cleavage site on the saccharide accumulated in lysosome of the patient.
(26) The method according to any one of (23) to (25), wherein the glycolytic enzyme is endo-type.
(27) The method according to any one of (23) to (26), wherein the lysosomal storage disease is selected from a group consisting of mucopolysaccharidosis, sphingolipidosis, glycogen storage disease type II, and glycoprotein storage disease.
(28) The method according to any one of (23) to (27), wherein the lysosomal storage disease is selected from a group consisting of Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, and mucopolysaccharidosis-plus syndrome.
(29) The method according to any one of (23) to (28), wherein the glycolytic enzyme is selected from a group consisting of glycosaminoglycan degrading enzyme, glycosidase, and peptide:N-glycanase.
(30) The method according to any one of (23) to (29), wherein the glycolytic enzyme is at least one selected from a group consisting of keratanase, heparinase, heparitinase, chondroitinase, hydaluronidase, β-galactosidase, α-galactosidase, PNGaseF, and endoglycosidase H.
(31) The method according to any one of (23) to (30), wherein the glycolytic enzyme is derived from a microorganism.
(32) The method according to (31), wherein the microorganism is a microorganism belonging to genus Bacillus.
(33) The method according to any one of (23) to (32), wherein the patient is a human.
(34) The method according to any one of (23) to (33), wherein a host cell producing the glycolytic enzyme is a microorganism.
(35) The method according to any one of (23) to (34), wherein the therapeutic agent is formulated into an injectable preparation.
(36) A method for treating lysosomal storage disease including:

a step of contacting blood taken from a patient with lysosomal storage disease with a glycolytic enzyme; and

a step of transporting the blood contacted with the glycolytic enzyme to the patient, and

the glycolytic enzyme does not have a mannose-6-phosphate moiety or a mannose moiety.

(37) The method according to (36), wherein the glycolytic enzyme is endo-type.
(38) The method according to (36) or (37), wherein the lysosomal storage disease is selected from a group consisting of mucopolysaccharidosis, sphingolipidosis, glycogen storage disease type II, and glycoprotein storage disease.
(39) The method according to any one of (36) to (38), wherein the lysosomal storage disease is selected from a group consisting of Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, and mucopolysaccharidosis-plus syndrome.
(40) The method according to any one of (36) to (39), wherein the glycolytic enzyme is selected from a group consisting of glycosaminoglycan degrading enzyme, glycosidase, and peptide:N-glycanase.
(41) The method according to any one of (36) to (40), wherein the glycolytic enzyme is at least one selected from a group consisting of keratanase, heparinase, heparitinase, chondroitinase, hydaluronidase, β-galactosidase, α-galactosidase, PNGaseF, and endoglycosidase H.
(42) The method according to any one of (36) to (41), wherein the glycolytic enzyme is derived from a microorganism.
(43) The method according to (42), wherein the microorganism is a microorganism belonging to genus Bacillus.
(44) The method according to any one of (36) to (43), wherein the patient is a human.
(45) The method according to any one of (36) to (44), wherein a host cell producing the glycolytic enzyme is a microorganism.
(46) The method according to any one of (36) to (45), wherein the therapeutic agent is formulated into an injectable preparation.

### EXAMPLES

Hereinbelow, preferred embodiments of the present invention are explained in more detail with reference to Examples. However, the technical scope of the present invention is not limited to the following Examples.

### Preparation Example

### (Method for Measuring Enzyme Activity)

The enzyme activity was measured by using Park-Johnson Method [J. Biol. Chem., 181, 149 (1949)] based on an increase of terminal reducing saccharide that is generated according to hydrolysis of keratan sulfate. Namely, to 50 μl (2.4 mg/ml) aqueous solution of keratan sulfate derived from bovine cornea, 50 μl of an enzyme solution and 100 μl of 0.2 M acetate buffer (pH 6.0) were added and the reaction was allowed to occur for 15 minutes at 37° C. By adding 200 μl of a carbonate-cyanide solution (5.3 g of sodium carbonate and 0.65 g of potassium cyanide were dissolved in 1000 ml of water) to the reaction solution, the reaction was terminated. Then, to the reaction solution, 200 μl of a ferricyanide solution (0.5 g of potassium ferricyanide dissolved in 1000 ml of water) was added and heated for 15 minutes in boiling bath. After cooling the reaction solution in water bath, 1 ml of a ferric sulfate solution (1.5 g of ammonium iron (III) sulfate dodecahydrate and 1 g of sodium dodecyl sulfate dissolved in 1000 ml of 0.05 N sulfuric acid) was added, followed by mixing. By allowing the reaction to occur for 15 minutes at 37° C., a measurement solution was obtained. Absorbance at 690 nm was measured using a spectrophotometer, and the obtained absorbance was taken as A. The absorbance which was obtained by carrying out the same treatment as above except that 50 μl of a heat-inactivated enzyme solution was used instead of the above enzyme solution was taken as A₀. Furthermore, the absorbance which was obtained by carrying out the same treatment as above except that 10 nmol/200 μl of N-acetylglucosamine solution was treated instead of the reaction solution is taken as Aₛₜ. The amount of the enzyme which produces a reducing saccharide corresponding to 1 μmol of galactose or N-acethylglucosamine during 1 minute at the above conditions was defined as 1 Unit (hereinbelow, it may be abbreviated as “Units” or “U”). The enzyme Unit number per 1 ml was calculated on the basis of the following equation.

$$1 Unit \text{/} ml = \frac{A - {A}_{0}}{{A}_{st}} \times \frac{\begin{matrix}[ Mole calibration ] \\ 10\end{matrix}}{1000} \times \frac{\begin{matrix}[ Dilution calibration ] \\ 1000\end{matrix}}{50} \times \frac{\begin{matrix}[ Time calibration ] \\ 1\end{matrix}}{15} \qquad [ Mathematical Formula 1 ]$$

### (Production and Purification of Glycolytic Enzyme)

Bacillus circulans KsT202 strain was subcultured with brain heart infusion slant medium (20 (w/v) % bovine brain hydrolyzate, 25 (w/v) % bovine heart hydrolyzate, 1 (w/v) % peptone digest product, 0.5 (w/v) % NaCl, 0.25 (w/v) % disodium phosphate, and 1.5 (w/v) % agar, pH 7.2) that were added with 0.2 (w/v) % keratan sulfate derived from shark cartilage.

20 L of a medium containing 1.5% (w/v) peptone (manufactured by Kyokuto Seiyaku Co., Ltd.), 0.75% (w/v) beer yeast extract (manufactured by Nihon Pharmaceutical Co., Ltd.), 0.75% (w/v) keratan sulfate (derived from shark cartilage, manufactured by Seikagaku Corporation), 0.5% (w/v) K₂HPO₄, 0.02% (w/v) MgSO₄.7H₂O, 0.5% (w/v) NaCl, and 0.0015% (w/v) antifoaming agent (ADEKA NOL™ LG109, manufactured by Asahi Denka Co., Ltd.) (pH 8.0) was prepared. The medium was added to a jar fermenter with volume of 30 L, and steam sterilization was carried out for 20 minutes at 121° C. Then, 1 L (5% (w/v)) of the cultured medium of Bacillus circulans KsT202 strain, which had been pre-cultured in the same medium under shaking for 16 hours at 37° C., was inoculated to the fresh medium sterilized above in a sterile manner. Then, culture was performed under stirring (300 rpm) for 24 hours at 45° C. under 1 vvm aeration rate. To remove cell bodies, the culture broth was centrifuged by the continuous solid-liquid separator, and approximately 20 L of extracellular fluid was obtained. The enzyme titer of keratanase included in this extracellular fluid was 11.6 mU/ml. Furthermore, Bacillus circulans KsT202 strain (accession number: FERM BP-5285) was deposited on Sep. 5, 1994 to the National Institute of Bioscience and Human Technology, Agency of Industrial Science and Technology, Ministry of International Trade and Industry (presently, Patent Organism Depositary Center, National Institute of Advanced Industrial Science and Technology, Independent Administrative Agency) with microorganism accession number of FERMP-14516. Furthermore, on Nov. 6, 1995, it was transferred to an international depositary organization based on Budapest Treaty. SEQ ID NO: 1 indicates the full-length amino acid sequence (GenBank: AAO88279.1) of keratanase (endo-β-N-acetylglucosaminidase) which is derived from Bacillus circulans KsT202 strain.

To obtain the protein from extracellular fluid, ammonium sulfate was added to give 70% saturation, and resulting precipitates were collected by centrifuge. The obtained precipitates were dissolved in 2.5 L of 10 mM Tris acetate buffer (pH 7.5, buffer A). To this solution, ammonium sulfate was added to give 35% saturation, and resulting precipitates were removed by centrifuge. To the supernatant, ammonium sulfate was added to give 55% saturation. Then, resulting precipitates were collected by centrifuge. The precipitates with 55% saturation were dissolved in 2.5 L of the buffer A (pH 7.5). Subsequently, the dissolved solution was allowed to pass through a DEAE-cellulose DE52 (manufactured by Whatman) column (5.2 cm×24 cm), which had been equilibrated in advance with the buffer A, to have enzyme adsorption. The column was washed with 1.5 L of the buffer A. Then, the sodium chloride concentration in the buffer A was linearly increased from 0 M to 0.3 M to elute the enzyme. The active fractions were collected and ammonium sulfate was added to give 55% saturation. Subsequently, the resulting precipitates were collected by centrifuge and dissolved in a small amount of 10 mM Tris acetate buffer (pH 7.5). Then, the dissolved solution was loaded onto a Sephacryl™ S-300 (manufactured by GE Healthcare) column (3.4 cm×110 cm), and then gel filtration chromatography was performed by using 50 mM Tris acetate buffer (pH 7.5) containing 0.5 M sodium chloride. The active fractions were concentrated by ultrafiltration with UK-10 membrane (manufactured by Advantec Toyo Kaisha, Ltd.) and dialyzed against the buffer A with an amount of approximately 100 times. The dialysis inner liquid was applied to DEAE-TOYOPEARL™ (manufactured by Tosoh Corporation) column (2.2 cm×15 cm), which has been equilibrated in advance with the buffer A. Subsequently, the column was washed with 150 ml of the buffer A containing 0.1 M NaCl. Then, the column was eluted with 0.1 M to 0.2 M NaCl linear gradient to obtain purified enzyme. The enzyme fractions were concentrated by ultrafiltration and loaded onto a Sephacryl™ S-300 (manufactured by GE Healthcare) column (2.2 cm×101 cm) to carry out gel filtration chromatography. NaCl were added the enzyme fractions to have 4 M NaCl solution. Then, the solution was applied to Phenyl Sepharose™ (manufactured by GE Healthcare) column (1.6 cm×15 cm), which had been equilibrated in advance with 10 mM Tris acetate (pH 7.5) containing 4 M NaCl. Subsequently, the column was eluted with the 4 M to 0 M NaCl linear gradient to obtain purified enzyme. 29 Units of enzyme was obtained, wherein the specific activity was 2.09 U/mg. The protein concentration was measured by Lowry assay using bovine serum albumin as a reference standard.

In order to lower endotoxin level, the enzyme obtained in above was applied to an Endo Trap™ HD column (ϕ0.7 cm×2.7 cm, 1 ml, Hyglos GmbH) equilibrated with 0.02 M HEPES buffer with 0.15 M NaCl and 0.1 mM CaCl₂, and flow-through fractions were collected. After use, the column was regenerated by 3 column volumes (CV) of 0.02 M HEPES buffer (pH 7.4) with 1 M NaCl and 2 mM ethylenediaminetetraacetic acid (EDTA). The endotoxin concentration in flow-through fractions was measured by ENDOSPECY™ (ES-50M, manufactured by Seikagaku Corporation Bio Business). This Endo Trap column operation was repeatedly carried out 8 times. The lastly recovered flow-through fraction was concentrated, buffer exchanged to phosphate buffered saline (PBS) with membrane ultrafiltration. The resultant solution was sterilized by 0.22 μm filtration. The final liquid amount was 9 ml, wherein the activity was 50.0 U/ml, the specific activity was 11.5 U/mg protein and endotoxin level was 12.8 Endotoxin Unit/mg protein. The protein concentration was measured by Lowry assay using bovine serum albumin as a reference standard.

### Example 1

### (Single Administration Study)

GALNS homo-knockout mice were used for the study. Eight-weeks-old GALNS homo-knockout mice were administered with keratanase at a single dose of 0.5 U/ml, 4 μl/g of the body weight via the tail vein (Treatment group). Alternatively, PBS was administered in the same manner (No-treatment group).

Serum samples (100 μl) were collected from the superficial temporal vein at the time points of 8 (before administration), 9, 10, 11, and 15-weeks-old of age.

TABLE 5

| | | Material for | Number of |
|---|---|---|---|
| Group | Genotype | administration | animals |
| No-treatment group | Galns−/− | PBS | 4 |
| Treatment group | Galns−/− | Keratanase/PBS | 5 |

### (Quantification of Serum Keratan Sulfate)

Quantification of keratan sulfate and heparin sulfate in serum was carried out based on the method described in the literature (Metabolites 2014, 4, 655-679). Briefly, keratan sulfate and heparin sulfate in the serum were digested by mixture of glycosaminoglycan degrading enzyme and the amount of generated disaccharides was quantified. The keratan mono-sulfated disaccharide standard, galactose β1→4N-acetylglucosamine-6-sulfate, was purchased from Carbosynth (product code, OA09703). The non-sulfated unsaturated heparan disaccharides (delta diHS-0S) standard was obtained from Seikagaku Corporation. The more detailed measurement method is described hereinbelow.

The serum sample was pre-treated according to the following method. To an Omega 10K membrane filter (PN8034, manufactured by Pall Corporation), 10 μl of a serum sample and 90 μl of 50 mM Tris hydrochloride buffer (pH 7.0) were added and low molecular weight components in the serum sample were removed by centrifugation at 2,500 g for 15 minutes. The membrane filter was set on a new reservoir plate, and to unused wells, each 10 μl of the serially diluted disaccharide standards was added. Ten microliter of internal control (chondrosine 5 μg/ml), 60 μl of 50 mM Tris hydrochloride solution (pH 7.0), and 0.5 mU/10 μl each of three glycosaminoglycan degrading enzyme were added to serum samples. These three enzymes were (i) chondroitinase ABC (derived from Proteus vulgaris, manufactured by Seikagaku Corporation) or chondroitinase B (derived from Flavobacterium heparinum, manufactured by Seikagaku Corporation), (ii) heparitinase (derived from Flavobacterium heparinum, manufactured by Seikagaku Corporation), and (iii) keratanase II (derived from Bacillus sp., manufactured by Seikagaku Corporation) solubilized into 50 mM Tris hydrochloride buffer. The plate was incubated for 16 hours at 37° C., and then centrifuged for 15 minutes at 2,500 g. The measurement sample collected on reservoir plate was stored at −20° C. until the quantification of keratan sulfate disaccharides.

For detection of keratan sulfate and heparan sulfate disaccharides, an LC-MS/MS system, 1210 Infinity LC system with 6460 triple quad mass spectrometer (manufactured by Agilent Technologies) was used. For separation of disaccharides, a Hypercarb™ column (P/N:35005-052130, ϕ2.0 mm×50 mm, 5 μm particles, manufactured by Thermo Scientific) was used. The mobile phase was gradient elution from 0 to 90% (v/v) acetonitrile in 0.025% (v/v) ammonia. Keratan sulfate mono-sulfated disaccharides were detected with precursor ion at 462.00 and product ion at 97.00 according to negative ion mode. Heparan sulfate non-sulfated disaccharides (delta diHS-0S) were detected with precursor ion at 379.29 and product ion at 175.10.

### (Results)

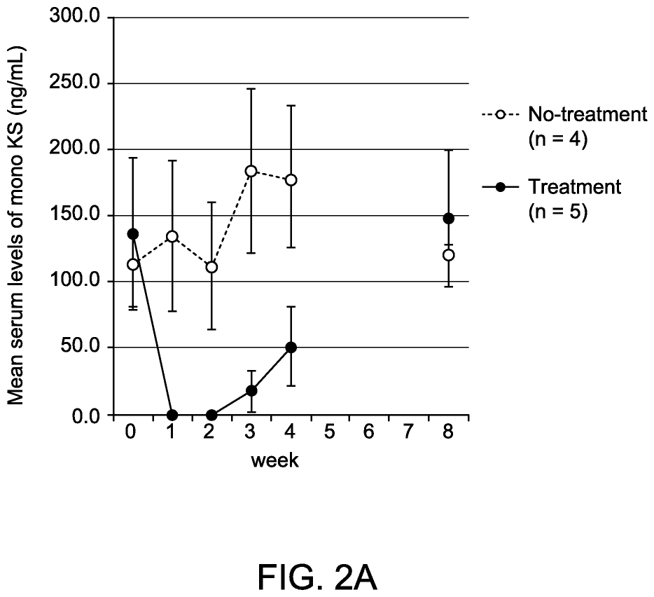

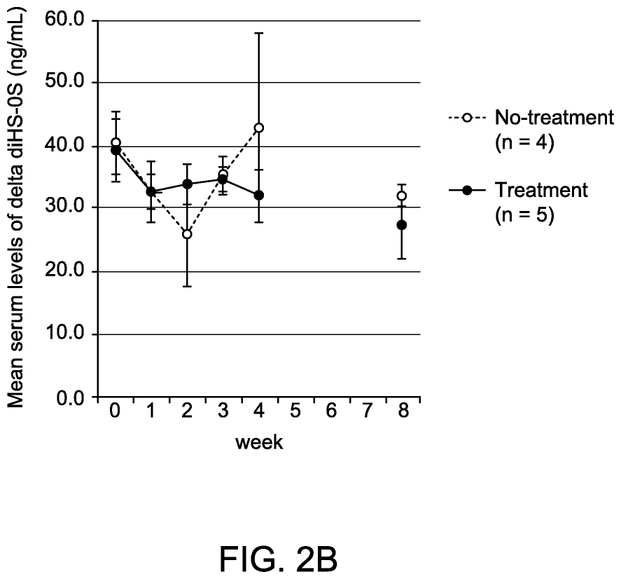

FIG. 2A shows the serum levels of keratan sulfate mono-sulfated disaccharide. The serum level of keratan sulfate mono-sulfated disaccharide was significantly reduced for more than 2 weeks by a single intravenous injection of the glycolytic enzyme derived from microorganism.

FIG. 2B shows the serum levels of delta diHS-0S. The serum level of delta diHS-0S was not affected by an intravenous injection of the glycolytic enzyme.

### Example 2

### (Repeated Administration Study)

GALNS homo- and hetero-knockout mice were used for the study. At 1 day or 2 days after birth, 80 mU/ml, 25 μl/g of the body weight of keratanase was administered to homo-knockout mice via the superficial temporal vein, and at 4- and 8-weeks of age, 0.5 U/ml, 4 μl/g of the body weight of keratanase was administered via the tail vein (Treatment group). Instead of keratanase, PBS was administered to GALNS homo- and hetero-knockout mice in the same manner (No-treatment group and Control group, respectively).

TABLE 6

| | | Material for | Number of |
|---|---|---|---|
| Group | Genotype | administration | animals |
| Control group | Galns^{+/-} | PBS | 10 |
| No-treatment group | Galns^{-/-} | PBS | 9 |
| Treatment group | Galns^{-/-} | Keratanase/PBS | 7 |

Serum samples (100 μl) were collected from the tail vein at 8- and 12-weeks-old of age. At 12-weeks-old of age, mice were sacrificed by carbon dioxide exposure and knee joints were collected for histopathology.

### (Histopathology)

The paraffin embedded tissue sections of knee joint were prepared as described below. Namely, the collected tissues were fixed with 10% (v/v) neutral buffered formalin solution. Subsequently, the tissues were decalcified with a decalcifying solution (formic acid:10% (v/v) neutral buffered formalin:distilled water=1:1:18 (v:v:v)) until the bones are softened. After completion of decalcification, the tissues were neutralized overnight with 5% (w/v) aqueous solution of sodium sulfate, and then rinsed in running water for 10 hours or so. Then, tissues were paraffin embedded and sectioned as following procedures; the tissues were dehydrated with ethanol series of 70% (v/v), 80% (v/v), and 99% (v/v) in order. Then, the ethanol was replaced with xylene, and the xylene was additionally replaced with paraffin wax. The paraffin wax infiltrated tissues were then embedded into wax blocks. 3 μm thick sections were made using a microtome. The sections were stained with hematoxylin and eosin (H&E).

The resin embedded tissue sections of knee joint were prepared as described below. The collected tissues were fixed with 2% (v/v) glutaraldehyde/4% (v/v) paraformaldehyde/phosphate buffer. Subsequently, the tissues were decalcified with a decalcifying solution (5% (w/v) EDTA2Na, pH 6.0 to 6.5) until the bones are softened. Then, tissues were fixed with osmium tetroxide, processed and embedded in a Spurr's resin. 0.5 μm thick sections were made using an ultra-microtome. The sections were stained with toluidine blue.

### (Results)

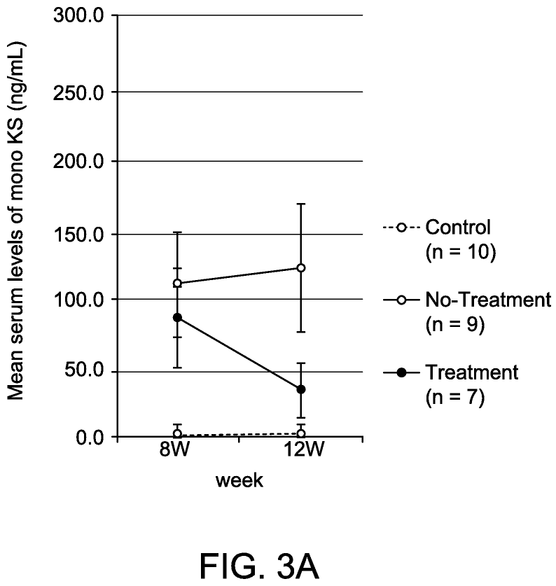

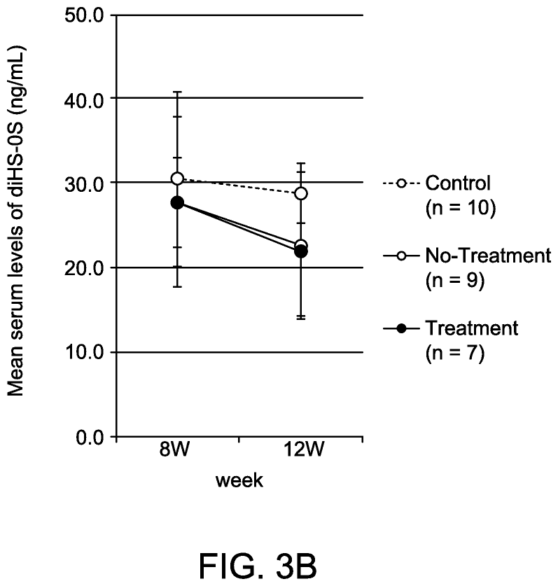

The result of the serum levels of keratan sulfate mono-sulfated disaccharide is shown in FIG. 3A. Serum levels of keratan sulfate mono-sulfated disaccharide in Treatment group were less than 30% of the No-treatment group at 12-weeks-old of age. On the contrary, the serum level of delta diHS-0S was not significantly affected as shown in FIG. 3B.

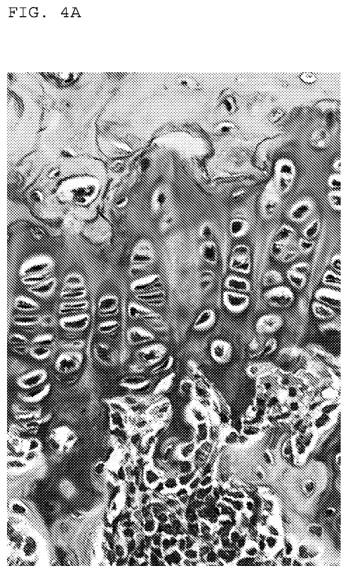

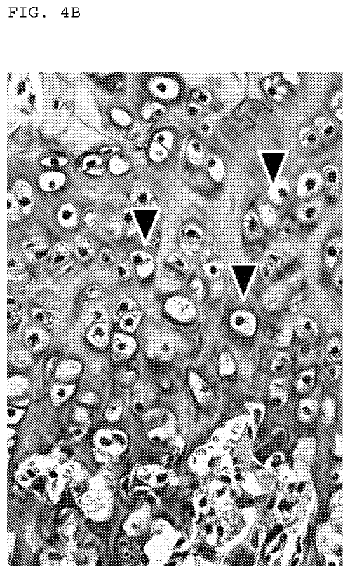

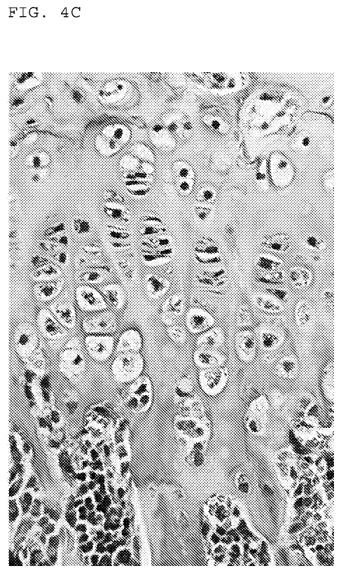

H&E stained sections of epiphyseal plate of femur from 12-weeks-old mice are shown in FIG. 4 (original magnification 400×). A mouse of No-treatment group (FIG. 4B) showed significant hypertrophy and vacuolation of the chondrocytes (arrow head) and disarrangement of chondrocytes compared to a mouse of Control group (FIG. 4A). On the other hand, in a mouse of Treatment group, hypertrophy and vacuolation of the chondrocytes and disarrangement of chondrocyte were suppressed compared to the mouse of No-treatment group (FIG. 4C).

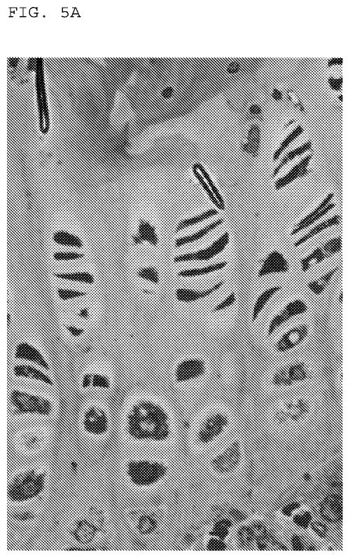

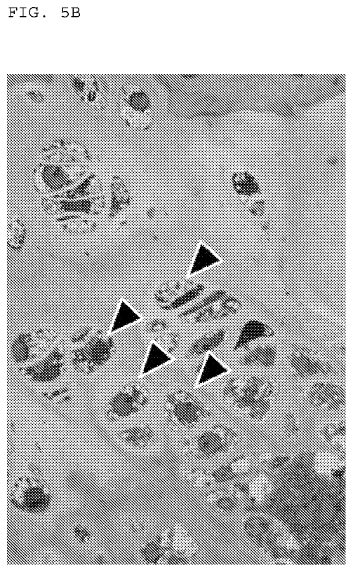

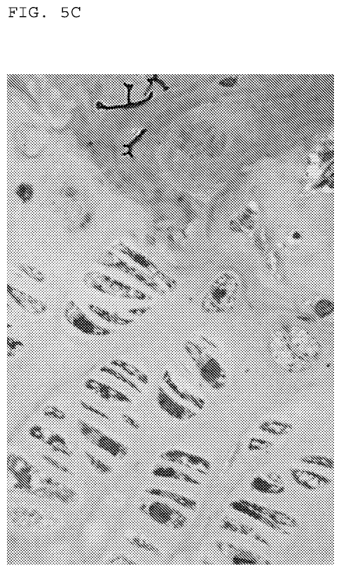

Toluidine blue stained sections of epiphyseal plate of femur from 12-weeks-old mice are shown in FIG. 5 (original magnification 800×). A mouse of No-treatment group (FIG. 5B) showed significant hypertrophy and vacuolation of the chondrocytes (arrow head) compared to the mouse of Control group (FIG. 5A). On the other hand, in a mouse of Treatment group, hypertrophy and vacuolation of the chondrocytes were suppressed compared to the mouse of No-treatment group (FIG. 5C).

### Example 3-1

### (Preparation of Enzyme Immobilized Column)

1.5 g of CNBr-activated Sepharose™ 4B (manufactured by GE Healthcare) is allowed to swell in 2.5 mM hydrochloric acid. On a glass filter, the Sepharose™ 4B resin is washed several times with 200 ml of 2.5 mM hydrochloric acid, and finally washed with 100 ml of the buffer B (pH 8.3) containing 0.1 M NaHCO₃ and 0.5 M NaCl. Against 2.4 ml of the swelled resin, 4 mg of keratanase which is obtained in the above is added and is suspended with 4 ml of solution B. The suspension is slowly stirred for 24 hours at low temperature (4° C.). After removing the pass-through on the glass filter, the resin is re-suspended in 0.2 M Tris hydrochloride (pH 8.0) with 10 times the resin volume. The suspension is slowly stirred for 24 hours at low temperature (4° C.). Then the resin is packed in a column, and is conditioned by the 3 CV of 0.1 M sodium acetate and 0.5 M NaCl (pH 4.0). Subsequently, the column is washed by the 3 CV of 0.1 M Tris hydrochloride with 0.5 M NaCl (pH 8.0). Finally, the column is washed by 25 ml PBS, and a glycolytic enzyme immobilized column is obtained.

### Example 3-2

### (Preparation of C-Terminal Truncated Keratanase)

C-terminal truncated keratanase gene (SEQ ID No. 2) was synthesized by GenScript and subcloned into BamHI and HindIII site of pET26b vector so that 6×His tag sequence could be added at C-terminal of an expression construct. The expression construct was transformed into E. coli BL21 Star (DE3) (Thermo Fisher Scientific). The transformant was cultured in LB medium with 30 μg/ml kanamycin at 37° C. until an OD₆₀₀ of 0.5 to 0.7. Isopropyl β-D-thiogalactopyranoside was added into the culture to give 0.4 to 1.0 mM final concentration and the culture was kept in a shaking incubator for 3 h at 37° C. The E. coli cells were harvested by centrifugation, and re-suspended in a liquid containing 10 mM Tris hydrochloride, 0.5 M NaCl and 1 mM phenylmethylsulfonyl fluoride. E. coli lysate, prepared by ultra-sonication and centrifugation at 50,000×g for 15 min at 4° C., was applied to Ni-sepharose column and an enzyme fraction was eluted with linear gradient 0 to 300 mM imidazole. In order to lowering endotoxins in the enzyme fraction, 5% (v/v) of Triton X-114 was mixed and the mixture was chilled on ice for 5 min. Then, the mixture was kept in 37° C. water bath for 5 min, followed by collecting upper phase by 2,300×g for 5 min. This step for lowering endotoxin was repeated for the five times. The enzyme solution was concentrated by ultrafiltration device (Amicon Ultra-15 100K, Merck Millipore) and loaded onto Hiprep™ 16/60 sephacryl column (GE healthcare) equilibrated with PBS. The resultant solution was sterilized by 0.22 μm filtration. 5 ml of liquid, having 11.3 U/ml of enzyme activity, and 1.5 U/mg of specific activity, and 0.003 Endotoxin Unit/mg protein of endotoxin level was finally obtained. The protein concentration was measured by micro BCA assay using bovine serum albumin as a reference standard.

### (Preparation of Enzyme Immobilized Column)

1 g of CNBr-activated sepharose 4B (manufactured by GE Healthcare) was allowed to swell in 2.5 mM hydrochloric acid. On a glass filter, the sepharose 4B resin was washed several times with 200 ml of 2.5 mM hydrochloric acid, and finally washed with 10 ml of the buffer B (pH 8.3) containing 0.1 M NaHCO₃ and 0.5 M NaCl. Approximately 4.5 mg of keratanase in 1.0 ml of buffer B obtained above was suspended to 1.0 ml of the swelled resin. Alternatively, 1.0 ml of the swelled resin was suspended with 1 ml of buffer B alone (control) or with 1 ml of buffer B containing 10 mg/mL bovine serum albumin (BSA). The suspensions were slowly stirred for 2 hours at ambient temperature. After removing a fraction passing-through a disposable empty column (Poly-Prep™ Chromatography Column, Bio-Rad), the resins were re-suspended in 10 ml of 0.2 M Tris hydrochloride (pH 8.0). The suspensions were slowly stirred for overnight at 4° C. Then, each 1 ml of un-immobilized (control), BSA-immobilized or keratanase-immobilized resin was packed respectively in Poly-Prep™ columns (bed height 2 cm). The packed columns were washed with the 3 CV of buffer B and 0.2 M Tris hydrochloride (pH 8.0) for 3 times so that residual free CNBr groups can be blocked, followed by washing with 5 CV of PBS. In this way, control column, BSA-immobilized column and keratanase-immobilized column were obtained.

In order to evaluate the efficacy of reducing the amount of keratan sulfate from the blood circulation, the columns were set at 35° C., and equilibrated with 3 CV of rabbit serum (CEDERLANE). Then, rabbit serum containing 5 mg/ml of keratan sulfate (derived from shark cartilage; manufactured by Seikagaku Corporation) was applied to the columns and eluent was sequentially fractionated every 1 ml into 3 tubes at flow rate of 40 cm/hour. The fractionated serum was diluted with water by ten-fold and immediately boiled for 5 min. Each 0.8 ml of sample was ultra-filtrated with centrifugal device (Nanosep™ with Omega 10K, PALL Life Sciences) and flow-through fraction was discarded. The retentate was washed once with 0.5 ml of water, and then dispensed to another tube and adjusted to 250 μl of volume with water.

### (Quantification of Keratan Sulfate in Immobilized Column Eluent)

Keratan sulfate content in the eluent from each column was quantified. Briefly, the retentate of column eluent was digested by keratanase II and the amount of generated disaccharides was quantified as follows:

One hundred microliters out of 250 μl retentate, 40 μl of 0.1 M sodium acetate (pH 6.0) and 10 μl of 1 mU keratanase II were mixed and incubated at 37° C. for 16 hours. The digested oligosaccharide was separated using Nanosep™ with Omega 10K (PALL Life Sciences) as flow-through. Then, 5 μl of flow through fraction was diluted with 5 μl of 10 μg/mL galactose-6-sulfate (Sigma Aldrich) solution, 5 μl of 1 M ammonium formate with 100 mM ammonium bicarbonate and 60 μl of acetonitrile.

For detection of keratan sulfate disaccharides, an LC-MS/MS system, ACQUITY UPLC I-Class system with Xevo TQ-XS mass spectrometer (manufactured by Waters) was used. For separation of disaccharides, a ACQUITY UPLC BEH Amide Column, 130 Å, 1.7 μm, 2.1 mm×100 mm (P/N: 186004801, Waters) was used. The mobile phase was a gradient elution from 50 to 90% (v/v) acetonitrile in 0.004% (v/v) ammonia containing 10 mM ammonium formate and 10 μM ammonium bicarbonate. Keratan sulfate mono-sulfated disaccharides were detected with precursor ion at 462.068 and product ion at 97 according to negative ion mode.

TABLE 7

| | Keratan sulfate mono-sulfate (μg) | | |
|---|---|---|---|
| Column | Fraction 1 | Fraction 2 | Fraction 3 |
| Control | 115.62 | 565.64 | 998.57 |
| (Un-Immobilized) | | | |
| BSA-Immobilized | 160.17 | 665.95 | 869.77 |
| Keratanase-Immobilized | 13.84 | 54.91 | 62.10 |

### Example 4

### (Measurement of Keratan Sulfate Levels in Mice Tissue)

GALNS homo-knockout mice were used for the study. Four-weeks-old GALNS homo-knockout mice were administered with keratanase at a single dose of 0.5 U/ml, 4 μl/g of the body weight via the tail vein (Treatment group). Instead of keratanase, PBS was administered in the same manner (No-treatment group). The mice were sacrificed at 24 hours after administration. Serum and tissue samples were collected and stored in deep-frozen until use. The frozen tissue was crushed with SK mill (freeze-crush apparatus, Tokken, Inc.) and digested by 12 U of thermolysin (SIGMA, T7902) in 200 mM ammonium acetate pH 8.1 with 5 mM CaCl₂ at 70° C. for 16 h. The solubilized sample was centrifuged at 20,000×g for 15 min, and the supernatant was mixed with 9 volumes of chilled ethanol and then kept 16 h at −20° C. Keratan sulfate was precipitated by centrifuge at 20,000×g for 15 min. The precipitate was reconstituted with 300 μl of distilled water and used for measurement of keratan sulfate level. The amount of keratan sulfate in serum and tissue samples were measured by LC-MS/MS systems in the same manner described in Example 1.

### (Results)

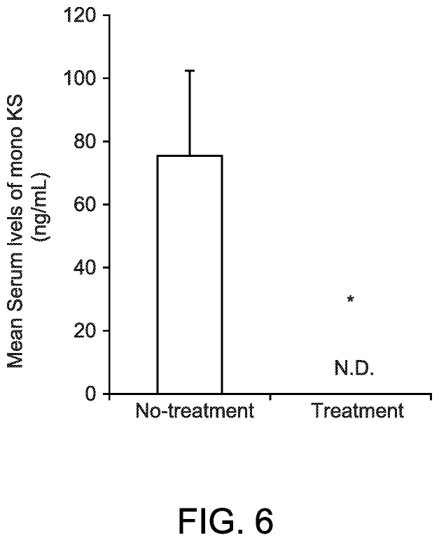

The result of the serum levels of keratan sulfate mono-sulfated disaccharide is shown in FIG. 6. Serum levels of keratan sulfate mono-sulfated disaccharide in Treatment group were significantly lower than No-treatment group.

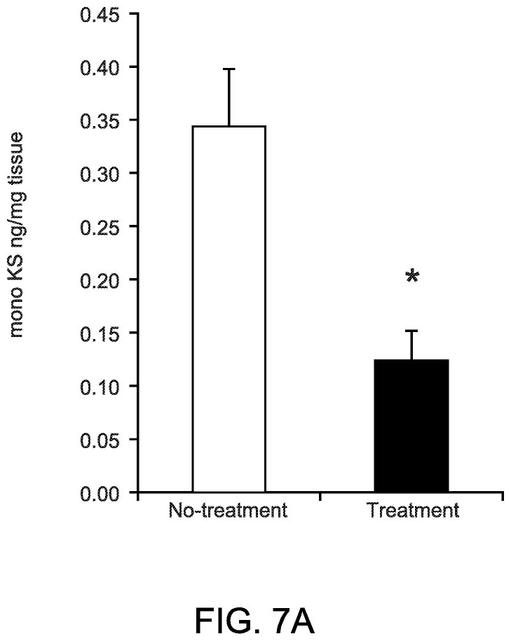

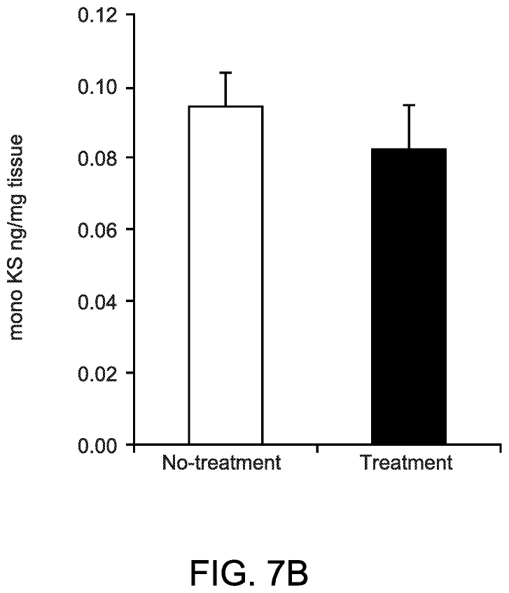

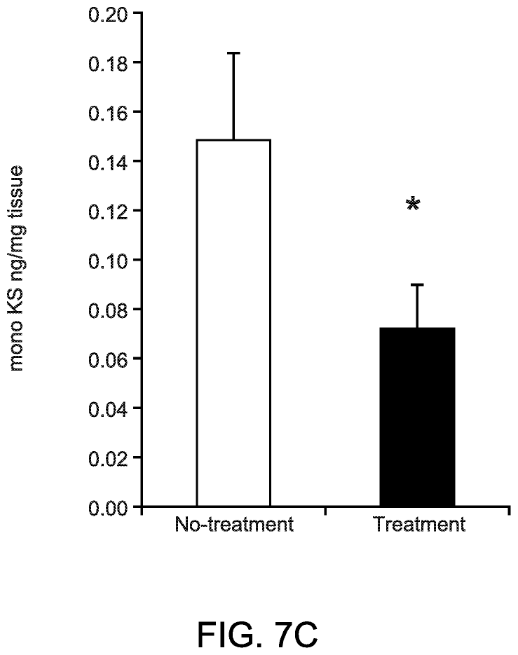

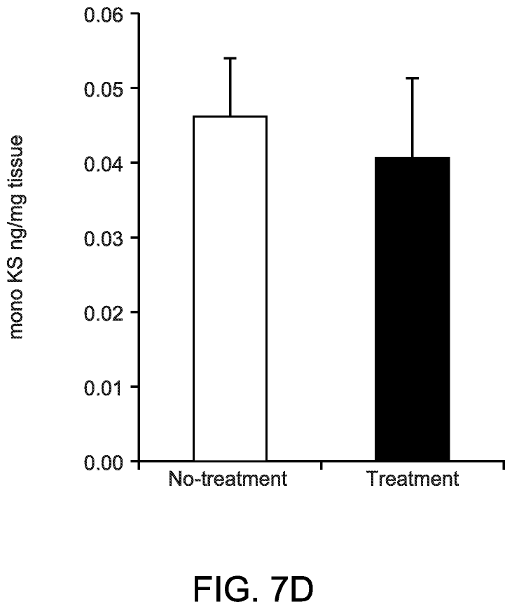

The amounts of keratan sulfate mono-sulfated disaccharide in several tissues are shown FIG. 7A to 7D. The keratan sulfate amounts of the liver and spleen in Treatment group were significantly lower than No-treatment group.

The documents cited in the present specification are incorporated herein by reference in their entirety.

While the invention has been described in connection with specific examples and various embodiments, it should be readily understood by the skilled in the art that many modifications and adaptations of the embodiments described herein are possible without departure from the spirit and scope of the invention. The description is intended to cover any variations, uses or adaptation of the invention, in general, the principles of the invention, and including such departures from the present disclosure as come within the known and customary practice within the art to which the invention pertains.

This application claims the benefit of priority to US Provisional Patent Application No. 62/556,076 filed on Sep. 8, 2017, U.S. Provisional Patent Application No. 62/556,644 filed on Sep. 11, 2017 and U.S. Provisional Patent Application No. 62/617,940 filed on Jan. 16, 2018, the entire contents of which are incorporated by reference herein.

### REFERENCE SIGNS LIST

1 Therapeutic device
2 Blood circuit
2a Blood sampling-side circuit
2b Blood reinfusing-side circuit
3 Blood pump
100 Therapeutic blood circulation system.

[Sequence Listing]

>AAO88279.1 endo-beta-N-acetylglucosaminidase [Bacillus
circulans] (SEQ ID NO: 1)
MSSRLKRKCSMLLTFTMIFQLLGLFLFKGEIVSASIRQDPTTGNYYKNVPLVGADFDDA
SNSNIVAKGTWDDNTAPLNTFFVDKGTATDGGFTTARITTVTDQVYEGGSLQFGDGSTY
PINLNYKVDGLEVGATYRLSAYMKLFPGYPAKGGQFGVKNHDTANYTTGGETKSVNFST
VTADWKEYSVTFTPTYPHAKIFFWGSNNLPKVLVDKLRLEKVLEHPGPAAPAVTADDVN
NIVVGIDETMEYNINGAGWVAYKEYAKPDLKGDLIVQIRVKETLNTLAGEVTTLTFTAQ
NDPAPGQPEQLLLKDGDFEAGAASVTTDTNVQNQFFSKNNQYEIVTGDTASGQYALKLR
SPETIGYHKTDLKPSTKYQISFMAKVGSASQKLSFRISGYKNDNPYDLDNVMNYIEHTQ
MKNTGWSRFYYDLETGPSATSAFIDFSTAAGSTAWIDDVKLVEQGPADPPVTEPTLSRG
SRLFLEKGLQIQSWVPTDVAYATRKWMKPPTAEEIVDLGLTTVQYNDAPNYSKTLHEEY
KKLQQTNPSLPDLKWGVAFGPNANHLSSSYFDSETIAKHDPNKTGAPTEEQKARGFLTP
DQLANVQNLNNIGFGDEEDYSDTLTQTLKEWFEVSKKHYPNVLVHHNEVGNTPPPTMSL
ISTFNENMLRKYMRTAKPDFITYDMYYFRENRQSSEVGGTVIPFYDDLNRYRKVASEGY
DGSGLSPIPFGTYLQGWRTGPGAATYEKRGDGWYEITESQAYLSAFANWTFGAKWLSMF
RWIEDTPGYLFSDYRPDEDGNWPKYHIYGQYKEMFRQSKNLGEHLIRINNKDVVIVPGQ
HMKDGQITKNNRPKDNPEWTKSGDRAFIDSLEISNLGKTNHSLKGDVFIGYFDPLPGID
TTQFFTSTAPKYFMLLNGLTSGQGLPAEEQTGSSYETRQEIKVTFDLSGGQARADQLRK
VSRLTGELVAAPLKDLGNGKYEMTVVLGGGMADLYFWELGSLNTGNSKPVVADTPHDVR
LTGDPKYAKNREIRDLTGKTVTVGWIKDTYSPVPQPLIHYNFSFTKDQNGKLQPMKNPD
ILSYFTRYYENTLWNKRVERIQKESNVKLEFVADIAWTKQELMDNIRKVKEGQTVDGMP
DILIVPDEWTWSGLIQNEMILPASSFSEFDFTERKWNKSYKAMTTWKDQIYGMYAGPTM
NSTGLFVNKALQASIGVTDDLMALQQNNAWDWNKLREVASAFQASANREGKYLLAGTDE
LFKQMVYANGAARGSVGGAMNQEFDLTSSSFREAAELYSELHAAGLIAAKPEGATDDWY
VEQFSKNNILFLALPYQQTVDKLKFSYTNQDAVIEMKEGSFLGQPALIPTIVDAYETAY
PDGIYKMAQGDWVFLMFPKGPSATGYAAMIDNPAYPVLLSSSANPADAAYVWNILSHEF
EGVAYDRFLKLYLNQREVDKTTLKRIGLKEGVWDSYSGTGAWEQVIKPGVLPMLQAGVI
DEAKLAELSVEAASYVTNNMTKPAQPGEEPGEEPGEQPGEQPGEQPGEQPGEQPGEQPG
EQPGEQPGEQPGAGNGSENQGGNEDQGGNGSQGGNGPKPEKIIVKPGELIAVEGKVTIV
VPAGATEIVLPPQAAELPQQHKVELKTDRVTLEVPSGLLKKLASRIADKDVSISLKAAP
LTAAQAKDAISKNKSVSPSAITLAGGVYDFKLSAAGANGSYAELSEFDQPITISLKIES
GVNPEQVGIYYISGNGKLDYIGGEYRDGELAAEVTHFSQYAVLKVVKVFDDVPAGHWAE
GVISKLTSRLMVDGTSETTFEPERVVTRAEFTALLARALKLTAGGTPTFADVKAGDWYA
DAVTAAVEAGIAEGKSAGQFEPQARITREEMVVMTMRAYNKAKDKGPSTGVEASFTDEN
QISAWAVEQVKAAAALQLIQGRAQGKFEPQGTATRAEAVQVIFNMLLK
//
>C-terminal truncated keratanase [Artificial Sequence] (SEQ ID
NO: 2)
SIRQDPTTGNYYKNVPLVGADFDDASNSNIVAKGTWDDNTAPLNTFFVDKGTATDGGFT
TARITTVTDQVYEGGSLQFGDGSTYPINLNYKVDGLEVGATYRLSAYMKLFPGYPAKGG
QFGVKNHDTANYTTGGETKSVNFSTVTADWKEYSVTFTPTYPHAKIFFWGSNNLPKVLV
DKLRLEKVLEHPGPAAPAVTADDVNNIVVGIDETMEYNINGAGWVAYKEYAKPDLKGDL
IVQIRVKETLNTLAGEVTTLTFTAQNDPAPGQPEQLLLKDGDFEAGAASVTTDTNVQNQ
FFSKNNQYEIVTGDTASGQYALKLRSPETIGYHKTDLKPSTKYQISFMAKVGSASQKLS
FRISGYKNDNPYDLDNVMNYIEHTQMKNTGWSRFYYDLETGPSATSAFIDFSTAAGSTA
WIDDVKLVEQGPADPPVTEPTLSRGSRLFLEKGLQIQSWVPTDVAYATRKWMKPPTAEE
IVDLGLTTVQYNDAPNYSKTLHEEYKKLQQTNPSLPDLKWGVAFGPNANHLSSSYFDSE
TIAKHDPNKTGAPTEEQKARGFLTPDQLANVQNLNNIGFGDEEDYSDTLTQTLKEWFEV
SKKHYPNVLVHHNEVGNTPPPTMSLISTFNENMLRKYMRTAKPDFITYDMYYFRENRQS
SEVGGTVIPFYDDLNRYRKVASEGYDGSGLSPIPFGTYLQGWRTGPGAATYEKRGDGWY
ElTESQAYLSAFANWTFGAKWLSMFRWIEDTPGYLFSDYRPDEDGNWPKYHIYGQYKEM
FRQSKNLGEHLIRINNKDVVIVPGQHMKDGQITKNNRPKDNPEWTKSGDRAFIDSLEIS
NLGKTNHSLKGDVFIGYFDPLPGIDTTQFFTSTAPKYFMLLNGLTSGQGLPAEEQTGSS
YETRQEIKVTFDLSGGQARADQLRKVSRLTGELVAAPLKDLGNGKYEMTVVLGGGMADL
YFWELGSLNTGNSKPVVADTPHDVRLTGDPKYAKNREIRDLTGKTVTVGWIKDTYSPVP
QPLIHYNFSFTKDQNGKLQPMKNPDILSYFTRYYENTLWNKRVERIQKESNVKLEFVAD
IAWTKQELMDNIRKVKEGQTVDGMPDILIVPDEWTWSGLIQNEMILPASSFSEFDFTER
KWNKSYKAMTTWKDQIYGMYAGPTMNSTGLFVNKALQASIGVTDDLMALQQNNAWDWNK
LREVASAFQASANREGKYLLAGTDELFKQMVYANGAARGSVGGAMNQEFDLTSSSFREA
AELYSELHAAGLIAAKPEGATDDWYVEQFSKNNILFLALPYQQTVDKLKFSYTNQDAVI
EMKEGSFLGQPALIPTIVDAYETAYPDGIYKMAQGDWVFLMFPKGPSATGYAAMIDNPA
YPVLLSSSANPADAAYVWNILSHEFEGVAYDRFLKLYLNQREVDKTTLKRIGLKEGVWD
SYSGTGAWEQVIKPGVLPMLQAGVIDEAKLAELSVEAASYVTNNMTKPAQPG
//
>Domain-C deleted keratanase [Artificial Sequence] (SEQ ID NO:
3)
SIRQDPTTGNYYKNVPLVGADFDDASNSNIVAKGTWDDNTAPLNTFYPHAKIFFWGSNN
LPKVLVDKLRLEKVLEHPGPAAPAVTADDVNNIVVGIDETMEYNINGAGWVAYKEYAKP
DLKGDLIVQIRVKETLNTLAGEVTTLTFTAQNDPAPGQPEQLLLKDGDFEAGAASVTTD
TNVQNQFFSKNNQYEIVTGDTASGQYALKLRSPETIGYHKTDLKPSTKYQISFMAKVGS
ASQKLSFRISGYKNDNPYDLDNVMNYIEHTQMKNTGWSRFYYDLETGPSATSAFIDFST
AAGSTAWIDDVKLVEQGPADPPVTEPTLSRGSRLFLEKGLQIQSWVPTDVAYATRKWMK
PPTAEEIVDLGLTTVQYNDAPNYSKTLHEEYKKLQQTNPSLPDLKWGVAFGPNANHLSS
SYFDSETIAKHDPNKTGAPTEEQKARGFLTPDQLANVQNLNNIGFGDEEDYSDTLTQTL
KEWFEVSKKHYPNVLVHHNEVGNTPPPTMSLISTFNENMLRKYMRTAKPDFITYDMYYF
RENRQSSEVGGTVIPFYDDLNRYRKVASEGYDGSGLSPIPFGTYLQGWRTGPGAATYEK
RGDGWYEITESQAYLSAFANWTFGAKWLSMFRWIEDTPGYLFSDYRPDEDGNWPKYHIY
GQYKEMFRQSKNLGEHLIRINNKDVVIVPGQHMKDGQITKNNRPKDNPEWTKSGDRAFI
DSLEISNLGKTNHSLKGDVFIGYFDPLPGIDTTQFFTSTAPKYFMLLNGLTSGQGLPAE
EQTGSSYETRQEIKVTFDLSGGQARADQLRKVSRLTGELVAAPLKDLGNGKYEMTVVLG
GGMADLYFWELGSLNTGNSKPVVADTPHDVRLTGDPKYAKNREIRDLTGKTVTVGWIKD
TYSPVPQPLIHYNFSFTKDQNGKLQPMKNPDILSYFTRYYENTLWNKRVERIQKESNVK
LEFVADIAWTKQELMDNIRKVKEGQTVDGMPDILIVPDEWTWSGLIQNEMILPASSFSE
FDFTERKWNKSYKAMTTWKDQIYGMYAGPTMNSTGLFVNKALQASIGVTDDLMALQQNN
AWDWNKLREVASAFQASANREGKYLLAGTDELFKQMVYANGAARGSVGGAMNQEFDLTS
SSFREAAELYSELHAAGLIAAKPEGATDDWYVEQFSKNNILFLALPYQQTVDKLKFSYT
NQDAVIEMKEGSFLGQPALIPTIVDAYETAYPDGIYKMAQGDWVFLMFPKGPSATGYAA
MIDNPAYPVLLSSSANPADAAYVWNILSHEFEGVAYDRFLKLYLNQREVDKTTLKRIGL
KEGVWDSYSGTGAWEQVIKPGVLPMLQAGVIDEAKLAELSVEAASYVTNNMTKPAQPG
//
>Domain-D deleted keratanase [Artificial Sequence] (SEQ ID NO:
4)
SIRQDPTTGNYYKNVPLVGADFDDASNSNIVAKGTWDDNTAPLNTFFVDKGTATDGGFT
TARITTVTDQVYEGGSLQFGDGSTYPINLNYKVDGLEVGATYRLSAYMKLFPGYPAKGG
QFGVKNHDTANYTTGGETKSVNFSTVTADWKEYSVTFTPTYPHAKIFFWGSNNLPKVLV
DKLRLEKVLEHPGPAQNDPAPGQPEQLLLKDGDFEAGAASVTTDTNVQNQFFSKNNQYE
IVTGDTASGQYALKLRSPETIGYHKTDLKPSTKYQISFMAKVGSASQKLSFRISGYKND
NPYDLDNVMNYIEHTQMKNTGWSRFYYDLETGPSATSAFIDFSTAAGSTAWIDDVKLVE
QGPADPPVTEPTLSRGSRLFLEKGLQIQSWVPTDVAYATRKWMKPPTAEEIVDLGLTTV
QYNDAPNYSKTLHEEYKKLQQTNPSLPDLKWGVAFGPNANHLSSSYFDSETIAKHDPNK
TGAPTEEQKARGFLTPDQLANVQNLNNIGFGDEEDYSDTLTQTLKEWFEVSKKHYPNVL
VHHNEVGNTPPPTMSLISTFNENMLRKYMRTAKPDFITYDMYYFRENRQSSEVGGTVIP
FYDDLNRYRKVASEGYDGSGLSPIPFGTYLQGWRTGPGAATYEKRGDGWYEITESQAYL
SAFANWTFGAKWLSMFRWIEDTPGYLFSDYRPDEDGNWPKYHIYGQYKEMFRQSKNLGE
HLIRINNKDVVIVPGQHMKDGQITKNNRPKDNPEWTKSGDRAFIDSLEISNLGKTNHSL
KGDVFIGYFDPLPGIDTTQFFTSTAPKYFMLLNGLTSGQGLPAEEQTGSSYETRQEIKV
TFDLSGGQARADQLRKVSRLTGELVAAPLKDLGNGKYEMTVVLGGGMADLYFWELGSLN
TGNSKPVVADTPHDVRLTGDPKYAKNREIRDLTGKTVTVGWIKDTYSPVPQPLIHYNFS
FTKDQNGKLQPMKNPDILSYFTRYYENTLWNKRVERIQKESNVKLEFVADIAWTKQELM
DNIRKVKEGQTVDGMPDILIVPDEWTWSGLIQNEMILPASSFSEFDFTERKWNKSYKAM
TTWKDQIYGMYAGPTMNSTGLFVNKALQASIGVTDDLMALQQNNAWDWNKLREVASAFQ
ASANREGKYLLAGTDELFKQMVYANGAARGSVGGAMNQEFDLTSSSFREAAELYSELHA
AGLIAAKPEGATDDWYVEQFSKNNILFLALPYQQTVDKLKFSYTNQDAVIEMKEGSFLG
QPALIPTIVDAYETAYPDGIYKMAQGDWVFLMFPKGPSATGYAAMIDNPAYPVLLSSSA
NPADAAYVWNILSHEFEGVAYDRFLKLYLNQREVDKTTLKRIGLKEGVWDSYSGTGAWE
QVIKPGVLPMLQAGVIDEAKLAELSVEAASYVTNNMTKPAQPG
//
>Domain-C, D deleted keratanase [Artificial Sequence] (SEQ ID NO:
5)
SIRQDPTTGNYYKNVPLVGADFDDASNSNIVAKGTWDDNTAPLNTFQNDPAPGQPEQLL
LKDGDFEAGAASVTTDTNVQNQFFSKNNQYEIVTGDTASGQYALKLRSPETIGYHKTDL
KPSTKYQISFMAKVGSASQKLSFRISGYKNDNPYDLDNVMNYIEHTQMKNTGWSRFYYD
LETGPSATSAFIDFSTAAGSTAWIDDVKLVEQGPADPPVTEPTLSRGSRLFLEKGLQIQ
SWVPTDVAYATRKWMKPPTAEEIVDLGLTTVQYNDAPNYSKTLHEEYKKLQQTNPSLPD
LKWGVAFGPNANHLSSSYFDSETIAKHDPNKTGAPTEEQKARGFLTPDQLANVQNLNNI
GFGDEEDYSDTLTQTLKEWFEVSKKHYPNVLVHHNEVGNTPPPTMSLISTFNENMLRKY
MRTAKPDFITYDMYYFRENRQSSEVGGTVIPFYDDLNRYRKVASEGYDGSGLSPIPFGT
YLQGWRTGPGAATYEKRGDGWYEITESQAYLSAFANWTFGAKWLSMFRWIEDTPGYLFS
DYRPDEDGNWPKYHIYGQYKEMFRQSKNLGEHLIRINNKDVVIVPGQHMKDGQITKNNR
PKDNPEWTKSGDRAFIDSLEISNLGKTNHSLKGDVFIGYFDPLPGIDTTQFFTSTAPKY
FMLLNGLTSGQGLPAEEQTGSSYETRQEIKVTFDLSGGQARADQLRKVSRLTGELVAAP
LKDLGNGKYEMTVVLGGGMADLYFWELGSLNTGNSKPVVADTPHDVRLTGDPKYAKNRE
IRDLTGKTVTVGWIKDTYSPVPQPLIHYNFSFTKDQNGKLQPMKNPDILSYFTRYYENT
LWNKRVERIQKESNVKLEFVADIAWTKQELMDNIRKVKEGQTVDGMPDILIVPDEWTWS
GLIQNEMILPASSFSEFDFTERKWNKSYKAMTTWKDQIYGMYAGPTMNSTGLFVNKALQ
ASIGVTDDLMALQQNNAWDWNKLREVASAFQASANREGKYLLAGTDELFKQMVYANGAA
RGSVGGAMNQEFDLTSSSFREAAELYSELHAAGLIAAKPEGATDDWYVEQFSKNNILFL
ALPYQQTVDKLKFSYTNQDAVIEMKEGSFLGQPALIPTIVDAYETAYPDGIYKMAQGDW
VFLMFPKGPSATGYAAMIDNPAYPVLLSSSANPADAAYVWNILSHEFEGVAYDRFLKLY
LNQREVDKTTLKRIGLKEGVWDSYSGTGAWEQVIKPGVLPMLQAGVIDEAKLAELSVEA
ASYVTNNMTKPAQPG
//

What is claimed is:

1. A method for treating lysosomal storage disease, comprising the step of administering a therapeutic agent comprising a glycolytic enzyme as an effective component,
wherein the glycolytic enzyme is different from a deficient protein of a patient with lysosomal storage disease as a subject and has an activity of degrading a saccharide accumulated in lysosome of the patient.

2. The method of claim 1, wherein the glycolytic enzyme does not have a mannose-6-phosphate moiety or a mannose moiety.

3. The method of claim 1, wherein the glycolytic enzyme is different from the deficient protein of the patient with lysosomal storage disease in terms of a cleavage site on the saccharide accumulated in lysosome of the patient.

4. The method of claim 1, wherein the glycolytic enzyme is endo-type.

5. The method of claim 1, wherein the lysosomal storage disease is selected from the group consisting of mucopolysaccharidosis, sphingolipidosis, glycogen storage disease type II, and glycoprotein storage disease.

6. The method of claim 1, wherein the lysosomal storage disease is selected from the group consisting of Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, and mucopolysaccharidosis-plus syndrome.

7. The method of claim 1, wherein the glycolytic enzyme is selected from the group consisting of glycosaminoglycan degrading enzyme, glycosidase, and peptide:N-glycanase.

8. The method of claim 1, wherein the glycolytic enzyme is at least one selected from the group consisting of keratanase, heparinase, heparitinase, chondroitinase, hydaluronidase, β-galactosidase, α-galactosidase, PNGaseF, and endoglycosidase H.

9. The method of claim 1, wherein the glycolytic enzyme is derived from a microorganism.

10. The method of claim 9, wherein the microorganism is a microorganism belonging to genus Bacillus.

11. The method of claim 1, wherein the patient is a human.

12. The method of claim 1, wherein a host cell producing the glycolytic enzyme is a microorganism.

13. The method of claim 1, wherein the therapeutic agent is formulated into an injectable preparation.

14. A method for producing a therapeutic agent for lysosomal storage disease containing a glycolytic enzyme as an effective component, the method comprising the steps of:
obtaining a culture of microorganism which produces the glycolytic enzyme;
collecting the glycolytic enzyme from the culture; and
optionally, formulating a composition by mixing the collected glycolytic enzyme with a pharmaceutically acceptable additive.

15. The method of claim 14, wherein the microorganism is selected from the group consisting of a microorganism belonging to genera Bacillus and Escherichia.

16. The method of claim 14, further comprising a step of lowering endotoxin level in the collected glycolytic enzyme to the extent that the endotoxin is substantially not contained.

17. A therapeutic device for lysosomal storage disease, comprising:
a carrier and a glycolytic enzyme immobilized to the carrier,
wherein the glycolytic enzyme is different from a deficient protein of a patient with lysosomal storage disease as a subject and has an activity of degrading a saccharide accumulated in lysosome of the patient.

18. A blood circulation system for treating lysosomal storage disease, comprising:
the therapeutic device of claim 17;
a blood sampling-side circuit for transporting blood taken from a patient with lysosomal storage disease to the therapeutic device;
a blood reinfusing-side circuit for transporting the blood contacted with the glycolytic enzyme comprised in the therapeutic device to the patient with lysosomal storage disease; and
a blood pump for pumping the blood through the blood sampling-side circuit and the blood reinfusing-side circuit.

19. A method for treating lysosomal storage disease, comprising the steps of:
contacting blood derived from a patient with lysosomal storage disease with a glycolytic enzyme; and
transporting the blood contacted with the glycolytic enzyme to the patient,
wherein the glycolytic enzyme is different from a deficient protein of the patient and has an activity of degrading a saccharide accumulated in lysosome of the patient.

20. The method of claim 19, wherein the glycolytic enzyme does not have a mannose-6-phosphate moiety or a mannose moiety.

21. The method of claim 19, wherein the glycolytic enzyme is different from the deficient protein of the patient with lysosomal storage disease in terms of a cleavage site on the saccharide accumulated in lysosome of the patient.

22. The method of claim 19, wherein the glycolytic enzyme is endo-type.

23. The method of claim 19, wherein the lysosomal storage disease is selected from the group consisting of mucopolysaccharidosis, sphingolipidosis, glycogen storage disease type II, and glycoprotein storage disease.

24. The method the claim 19, wherein the lysosomal storage disease is selected from the group consisting of Hurler disease, Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease, mucopolysaccharidosis type IX, and mucopolysaccharidosis-plus syndrome.

25. The method claim 19, wherein the glycolytic enzyme is selected from the group consisting of glycosaminoglycan degrading enzyme, glycosidase, and peptide:N-glycanase.

26. The method of claim 19, wherein the glycolytic enzyme is at least one selected from the group consisting of keratanase, heparinase, heparitinase, chondroitinase, hydaluronidase, β-galactosidase, α-galactosidase, PNGaseF, and endoglycosidase H.

27. The method of claim 19, wherein the glycolytic enzyme is derived from a microorganism.

28. The method of claim 27, wherein the microorganism is a microorganism belonging to genus Bacillus.

29. The method of claim 19, wherein the patient is a human.

30. The method of claim 19, wherein a host cell producing the glycolytic enzyme is a microorganism.

31. The method of claim 19, wherein the therapeutic agent is formulated into an injectable preparation.
